SEQUENCE LISTING (17 sequences)

SEQ ID NO: 1 - protein, 1416 aa, Artificial Sequence
  SYNTHETIC POLYPEPTIDE
MGSSAPKKKRKVGIHGVPAAMDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAAPKKKRKVDPKKKRKVAAALEHHHHHH

SEQ ID NO: 2 - RNA, 36 nt, Artificial Sequence
  SYNTHETIC POLYNUCLEOTIDE
  misc_feature (1)..(1): 5′-C3 spacer
  misc_feature (36)..(36): 3′-C3 spacer
cuuauauccaacacuucgugguuuuagagcuaugcu

SEQ ID NO: 3 - RNA, 67 nt, Artificial Sequence
  SYNTHETIC POLYNUCLEOTIDE
  misc_feature (1)..(9): 2-OMethyl modified ribose moiety
  misc_feature (2)..(3): Phosphorothioate internucleotide modification
  misc_feature (38)..(67): 2-OMethyl modified ribose moiety
  misc_feature (66)..(67): Phosphorothioate internucleotide modification
agcauagcaaguuaaaauaaggcuaguccguuaucaacuugaaaaaguggcaccgagucggugcuuu

SEQ ID NO: 4 - DNA, 1083 bp, Artificial Sequence
  SYNTHETIC POLYNUCLEOTIDE
aagaatgttgtgataaaaggtgatgctcacctctcccacacccttttatagtttagggattgtatttccaaggtttctagactgagagcccttttcatctttgctcattgacactctgtacccattaatcctccttattagctccccttcaatggacacatgggtagtcagggtgcaggtctcagaactgtccttcaggttccaggtgatcaaccaagtgccttgtctgtagtgtcaactcattgctgccccttcctagtaatccccataatttagctctccatttcatagtctttccttgggtgtgttaaaagtgaccatggtacactcagcacggatgaaatgaaacagtgtttagaaacgtcagtcttctcttttgtaatgccctgtagtctctctgtatgttatatgtcacattttgtaattaacagcttgctggtgaaaaggaccccacgaagtgttggatataagccagactgtaagtgaattactttttttgtcaatcatttaaccatctttaacctaaaagagttttatgtgaaatggcttataattgcttagagaatatttgtagagaggcacatttgccagtattagatttaaaagtgatgttttctttatctaaatgatgaattatgattctttttagttgttggatttgaaattccagacaagtttgttgtaggatatgcccttgactataatgaatacttcagggatttgaatgtaagtaattgcttctttttctcactcatttttcaaaacacgcataaaaatttaggaaagagaattgttttctccttccagcacctcataatttgaacagactgatggttcccattagtcacataaagctgtagtctagtacagacgtccttagaactggaacctggccaggctagggtgacacttcttgttggctgaaatagttgaacagctttaatatacaataattgttgcattattatttcagatgataaatgtggtcataagtaagaaataaatgatcgagtttagtcttttaattcactgtcctttgaatacctgcctcttactctggaggcagaagtcccatggatgtgt

SEQ ID NO: 5 - DNA, 27 bp, Artificial Sequence
  SYNTHETIC POLYNUCLEOTIDE
aagaatgttgtgataaaaggtgatgct

SEQ ID NO: 6 - DNA, 24 bp, Artificial Sequence
  SYNTHETIC POLYNUCLEOTIDE
acacatccatgggacttctgcctc

SEQ ID NO: 7 - RNA, 36 nt, Artificial Sequence; identical to SEQ ID NO: 2
  SYNTHETIC POLYNUCLEOTIDE
  misc_feature (1)..(1): 5′-C3 Spacer
  misc_feature (36)..(36): 3′ ATTO550, coupled as NHS-ester dye to amino-mod RNA

SEQ ID NO: 8 - RNA, 36 nt, Artificial Sequence; identical to SEQ ID NO: 2
  SYNTHETIC POLYNUCLEOTIDE
  misc_feature (1)..(1): 5′-C3 Spacer
  misc_feature (36)..(36): 3′ ATTO647, coupled as NHS-ester dye to amino-mod RNA

SEQ ID NO: 9 - RNA, 36 nt, Artificial Sequence; identical to SEQ ID NO: 2
  SYNTHETIC POLYNUCLEOTIDE
  misc_feature (1)..(1): 5′-C3 Spacer
  misc_feature (36)..(36): 3′ ATTO655, coupled as NHS-ester dye to amino-mod RNA

SEQ ID NO: 10 - RNA, 36 nt, Artificial Sequence; identical to SEQ ID NO: 2
  SYNTHETIC POLYNUCLEOTIDE
  misc_feature (1)..(1): 5′-C3 Spacer
  misc_feature (36)..(36): 3′ Cy3, coupled as NHS-ester dye to amino-mod RNA

SEQ ID NO: 11 - RNA, 36 nt, Artificial Sequence; identical to SEQ ID NO: 2
  SYNTHETIC POLYNUCLEOTIDE
  misc_feature (1)..(1): 5′-C3 Spacer
  misc_feature (36)..(36): 3′ Cy5, coupled as NHS-ester dye to amino-mod RNA

SEQ ID NO: 12 - RNA, 36 nt, Artificial Sequence
  SYNTHETIC  POLYNUCLEOTIDE
  misc_feature (1)..(1): 5′-C3 Spacer
  misc_feature (36)..(36): 3′-C3 Spacer
aauuauggggauuacuaggaguuuuagagcuaugcu

SEQ ID NO: 13 - RNA, 67 nt, Artificial Sequence; identical to SEQ ID NO: 3
  SYNTHETIC POLYNUCLEOTIDE
  misc_feature (1)..(1): 5′ ATTO550, coupled as NHS-ester dye to amino-mod RNA
  misc_feature (1)..(9): 2′-OMethyl ribose moiety
  misc_feature (2)..(3): Phosphorothioate internucleotide modification
  misc_feature (38)..(67): 2′-OMethyl ribose moiety
  misc_feature (66)..(67): Phosphorothioate internucleotide modification

SEQ ID NO: 14 - RNA, 67 nt, Artificial Sequence; identical to SEQ ID NO: 3
  SYNTHETIC POLYNUCLEOTIDE
  misc_feature (1)..(1): 5′ ATTO647, coupled as NHS-ester dye to amino-mod RNA
  misc_feature (1)..(9): 2′-OMethyl ribose moiety
  misc_feature (2)..(3): Phosphorothioate internucleotide modification
  misc_feature (38)..(67): 2′-OMethyl ribose moiety
  misc_feature (66)..(67): Phosphorothioate internucleotide modification

SEQ ID NO: 15 - RNA, 67 nt, Artificial Sequence; identical to SEQ ID NO: 3
  SYNTHETIC POLYNUCLEOTIDE
  misc_feature (1)..(1): 5′ ATTO655, coupled as NHS-ester dye to amino-mod RNA
  misc_feature (1)..(9): 2′-OMethyl ribose moiety
  misc_feature (2)..(3): Phosphorothioate internucleotide modification
  misc_feature (38)..(67): 2′-OMethyl ribose moiety
  misc_feature (66)..(67): Phosphorothioate internucleotide modification

SEQ ID NO: 16 - RNA, 67 nt, Artificial Sequence; identical to SEQ ID NO: 3
  SYNTHETIC POLYNUCLEOTIDE
  misc_feature (1)..(1): 5′ Cy3, coupled as NHS-ester dye to amino-mod RNA
  misc_feature (1)..(9): 2′-OMethyl ribose moiety
  misc_feature (2)..(3): phosphorothioate internucleotide modification
  misc_feature (38)..(67): 2′-OMethyl ribose moiety
  misc_feature (66)..(67): phosphorothioate internucleotide modification

SEQ ID NO: 17 - RNA, 67 nt, Artificial Sequence; identical to SEQ ID NO: 3
  SYNTHETIC POLYNUCLEOTIDE
  misc_feature (1)..(1): 5′ ATTO488, coupled as NHS-ester dye to amino-mod RNA
  misc_feature (1)..(9): 2′-OMethyl ribose moiety
  misc_feature (2)..(3): Phosphorothioate internucleotide modification
  misc_feature (38)..(67): 2′-OMethyl ribose moiety
  misc_feature (66)..(67): Phosphorothioate internucleotide modification

(19) United States
(12) Patent Application Publication
(10) Pub. No.: US 2019/0032131 A1
(43) Pub. Date: Jan. 31, 2019

(54) GENOME EDITING DETECTION

(71) Applicant: Integrated DNA Technologies, Inc., Coralville, IA (US)

(72) Inventors: Rolf Turk, Iowa City, IA (US); Ashley Jacobi, North Liberty, IA (US); Mark A. Behlke, Coralville, IA (US)

(21) Appl. No.: 15/839,817

(22) Filed: Dec. 12, 2017

Related U.S. Application Data
(60) Provisional application No. 62/432,787, filed on Dec. 12, 2016.

(51) Int. Cl. C12Q 1/6876; C12N 15/11; C12N 9/22; G01N 21/64

(52) U.S. Cl. CPC C12Q 1/6876; C12N 15/11; C12N 9/22; G01N 2021/6439; C12N 2310/20; C12N 2800/80; G01N 21/6428

(57) ABSTRACT

This invention pertains to labeled components of a guide RNA complex as part of the CRISPR/Cas9 editing complex in order to detect and visualize successful delivery of the CRISPR/Cas9 editing complex.

### CROSS-REFERENCE TO RELATED APPLICATIONS

This application claims benefit of priority under 35 U.S.C. 119 to U.S. Provisional Patent Application Ser. No. 62/432,787, filed Dec. 12, 2016 and entitled “GENOME EDITING DETECTION,” the contents of which are herein incorporated by reference in their entirety.

### SEQUENCE LISTING

The instant application contains a Sequence Listing that has been submitted in ASCII format via EFS-Web and is hereby incorporated by reference in its entirety. The ASCII copy, created on______ , is named IDT01-012-US_ST25.txt, and is______ bytes in size.

### FIELD OF THE INVENTION

This invention pertains to labeled components of a guide RNA complex as part of the CRISPR/Cas9 editing complex in order to detect and visualize successful delivery of the CRISPR/Cas9 editing complex, localized said complexes within a cell, as well as to enrich cell populations for cells that have taken up the complex.

### BACKGROUND OF THE INVENTION

The recently discovered bacterial CRISPR/Cas9 system is used to generate editing events in double-stranded DNA. The system relies on the nuclease activity of Cas9, which activity leads to double-stranded breaks (DSBs), as well as a guide RNA that directs the Cas9 protein to a specific sequence-dependent location (see Jinek et al., Science (2012) 337:816-821). The CRISPR/Cas9 system has been successfully used to alter genomic DNA in different model systems as well as in various organisms (see Harms et al., Curr Protoc Hum Genet (2015) 83:15.7.1-15.7.27). The generation of DSBs in genomic DNA leads to activation of either the non-homologous end-joining (NHEJ) pathway or, when a template with homologous arms to the cut site is present, the homology-directed repair pathway (HDR). As a result, the CRISPR/Cas9 system is widely used to alter genomic DNA sequences.

Both the Cas9 endonuclease and CRISPR guide RNAs (gRNAs) must be present in a cell for DNA cleavage to occur. The CRISPR/Cas9 components can be introduced into the cell using various approaches. Examples include plasmid or viral expression vectors (which lead to endogenous expression of either Cas9, the gRNAs, or both), Cas9 mRNA with separate gRNA transfection, or delivery of Cas9 protein with the guide RNAs as a ribonucleoprotein (RNP) complex (See Kouranova et al., Hum Gen Ther (2016) 27(6):464-475). Due to a relatively quick turn-over of the RNP complex compared to plasmid or viral expression vectors, the amount of off-target effects is low (see Liang et al., J Biotech (2015) 208:44-53). Therefore, performing genome editing by delivery of the RNP complex is often a preferred method.

The ribonucleoprotein complex (RNP) can be delivered to cells using different transfection methods. Lipofection relies on complexation of the RNP with cationic lipids, and has the potential to reach high levels of editing efficiency (see Yu et al., Biotechnol Lett (2016) 38:919-929). The methodology is straight-forward, but has a number of disadvantages. First, cationic lipids can be toxic to cells when administered at high concentrations. Second, many cell types, including primary human cells which have the greatest interest for medical application, cannot be transfected using traditional cationic lipids. Third, the size and polarity of Cas9 leads to complexation issues as the cationic lipids do not bind well to the cationic regions of the proteins. An alternative to lipofection is electroporation. The RNP is delivered into the cell by diffusion after pores in the cell membrane are created by applying a cell-specific current. High levels of genome editing can be achieved, but require relative high concentrations of RNP. The electroporation methodology is recommended for hard-to-transfect cell lines and primary cells. With electroporation, there is often a trade-off between effectiveness of RNP delivery with cell survival, where conditions that are favorable for delivery often kill an increasing fraction of the cells. To keep cells healthy, it may be necessary to employ suboptimal electroporation conditions for delivery, so a fraction of the cell population will not have been transfected, but the cells are healthier. A method to separate transfected from non-transfected cells (enrich for cells with RNP) would be useful for both research and therapeutic applications.

The invention provides such a method. These and other advantages of the invention, as well as additional inventive features, will be apparent from the description of the invention provided herein.

### BRIEF SUMMARY OF THE INVENTION

The invention provides compositions and methods of use of fluorescently-labeled guide RNA components to detect, visualize and, additionally, to select and enrich for cells successfully transfected with ribonucleoprotein having CRISPR-associated endonuclease editing activity.

In a first aspect, a method of detecting cells containing a CRIPSR ribonucleoprotein complex is disclosed. The method includes a first step of contacting a CRISPR-associated RNA and a CRISPR-associated protein with a cell sample. The CRISPR-associated RNA is conjugated to a label configured to generate a signal. The CRISPR-associated protein is Cas9 protein or Cpfl protein. The method includes a second step of determining a presence of the signal from at least one cell in the cell sample. The presence of the signal from at least one cell in the cell sample detects at least one cell containing the CRIPSR ribonucleoprotein complex.

In a second aspect, a nucleic acid conjugate comprising a CRISPR-associated RNA conjugated to a label configured to generate a signal is provided. The CRISPR-associated RNA is selected from the group consisting of a crRNA and a tracrRNA.

In a third aspect, a kit for detecting cells containing a CRISPR ribonucleoprotein complex is provided. In preferred embodiments, the kit includes a nucleic acid conjugate. The nucleic acid conjugate includes an RNA conjugated to a label configured to generate a signal, The RNA is selected from the group consisting of a gRNA, a crRNA and a tracrRNA.

### BRIEF DESCRIPTION OF THE DRAWINGS

FIG. 1 shows an example of an aligned crRNA and tracrRNA used in this study. In this case, both the crRNA (SEQ ID NO:2) and tracrRNA (SEQ ID NO:3) are unlabeled in that they don't contain any attached fluorophores. The 20 base 5′-domain (boldface) in the crRNA is the target specific protospacer sequence which varies with each different target sequence and, in this case, targets human HPRT1 at position 38285-AS. 3′-domain of the crRNA binds to a region towards the 5′-end of the tracrRNA. The crRNAs and tracrRNAs used in this study have shorter lengths than those found endogenously in Streptococcus pyogenes. Modifications, such as phosphorothioate linkages and 2′OMe RNA, were omitted for clarity.

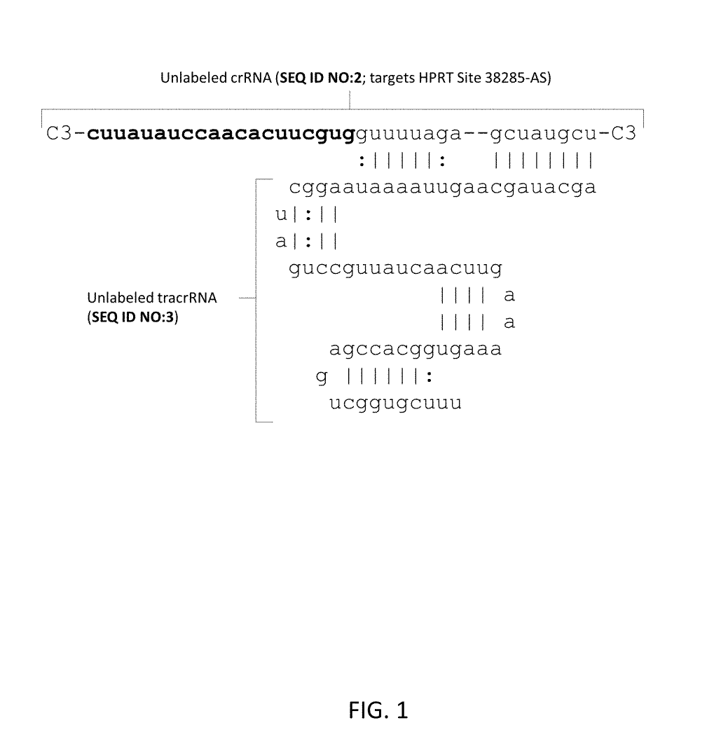

FIG. 2 shows (1) the native human hypoxanthine phosphoribosyl transferase (HPRT1) sequence used in the present study (SEQ ID NO:4), (2) the locations and sequences of the two target sites used (at positions 38285-AS and 38087-AS), and (3) the priming sites for the forward and reverse HPRT1 primers (SEQ ID NOs:5-6) used for the amplification step prior to the T7EI cleavage analysis. The protospacer sequence for the target sites at positions 38285-AS and 38087-AS is on the strand opposite that of HPRT1 gene. Therefore, the reverse complements of the protospacers shown in FIG. 2. The three-letter reverse complement PAM sequences are underlined and in boldface.

FIG. 3A shows the T7EI editing efficiencies of labeled guide RNA complexes introduced into HEK393 cells using lipofection via RNAiMAX. Labeled crRNAs (SEQ ID NOs:7-11) were complexed to unlabeled tracrRNAs (SEQ ID NO:3), whereas labeled tracrRNAs (SEQ ID NOs:13-17) were complexed to unlabeled crRNAs (SEQ ID NOs:2 and 12). In all cases, the crRNA and tracrRNA were complexed at a 1:1 molar ratio. The resulting guide RNA complexes were then bound to Cas9 protein (SEQ ID NO:1) as RNP at a 1:1 molar ratio and transfected into HEK293 cells via lipofection. The final RNP concentration was 10 nM. After a 48-hour incubation, genomic DNA was isolated, the target region amplified via PCR and digested with 2 U T7 endonuclease I (T7EI). The percent editing was determined by fragment separation on the Fragment Analyzer.

FIG. 3B shows the T7EI editing efficiencies of labeled guide RNA complexes introduced into HEK393 cells using electroporation with the Amaxa Nucleofector 96-well Shuttle System (Lonza). The final RNP concentration was either 0.5 or 4 μM and electroporation into HEK293 cells was carried out in the presence of 4 μM Alt-R Electroporation Enhancer. Cell density was 3.5E5 cells/electroporation. Unlabeled crRNA was specific to the HPRT1 gene at position 38285-AS (SEQ ID NO:2) or at position 38087-AS (SEQ ID NO:12). Either unlabeled tracrRNA (SEQ ID NO:3), ATTO550-labeled tracrRNA (SEQ ID No.13), or ATTO-488-labeled tracrRNA (SEQ ID NO:17) was used. After a 48-hour incubation, the genomic DNA was isolated, the target region amplified via PCR and digested with 2.5 U T7EI endonuclease. The percent editing was determined by fragment separation on a Fragment Analyzer.

FIG. 4 illustrates the cytometric resolution of positively transfected cells. Jurkat cells were electroporated with RNP consisting of either labeled crRNA or labeled tracrRNA. The ratio of gRNA:Cas9 protein:Alt-R® Cas9 Electroporation Enhancer was 1.2:1:1. Jurkat cells subjected to RNP but without electroporation were used as background controls. Gates were set to not include the background controls. The percentage of cells within the gate settings was determined (Positive Cell Fraction).

FIG. 5A illustrates the effect of washing cells prior to cytometric resolution of Jurkat cells electroporated with RNP consisting of a final concentration of Cas9 protein at 1.5 or 0.15 μM with unlabeled crRNA and ATTO550-labeled tracrRNA. The ratio of gRNA:Cas9 protein:Alt-R® Cas9 Electroporation Enhancer was 1.2:1:1. Jurkat cells subjected to RNP but without electroporation were used as background controls. Gates were set to not include the background controls. Cells were sorted 24 hours post-transfection. Prior to sorting, cells were washed 0, 1 or 2 times with PBS +1% FBS. Histogram plots show fluorescence intensities for ATTO550.

FIG. 5B illustrates the positive cell fraction and mean fluorescence intensity of Jurkat cells electroporated with RNP consisting of a final concentration of Cas9 protein at 1.5 or 0.15 μM with unlabeled crRNA and ATTO550-labeled tracrRNA as a function of washes according to the experimental details as described in FIG. 5A. The percentages of cells within the gate settings were determined and expressed as positive cell fraction. The mean fluorescence intensity (MFI) was determined by subtracting the respective background control from each sample for ATTO550.

FIG. 5C illustrates the effect of washing cells prior to cytometric resolution of Jurkat cells electroporated with RNP consisting of a final concentration of Cas9 protein at 1.5 or 0.15 μM with unlabeled crRNA and ATTO647-labeled tracrRNA. The ratio of gRNA:Cas9 protein:Alt-R® Cas9 Electroporation Enhancer was 1.2:1:1. Jurkat cells subjected to RNP but without electroporation were used as background controls. Gates were set to not include the background controls. Cells were sorted 24 hours post-transfection. Prior to sorting, cells were washed 0, 1 or 2 times with PBS+1% FBS. Histogram plots show fluorescence intensities for ATTO647.

FIG. 5D illustrates the positive cell fraction and mean fluorescence intensity of Jurkat cells electroporated with RNP consisting of a final concentration of Cas9 protein at 1.5 or 0.15 μM with unlabeled crRNA and ATTO647-labeled tracrRNA as a function of washes according to the experimental details as described in FIG. 5C. The percentages of cells within the gate settings were determined and expressed as positive cell fraction. The mean fluorescence intensity (MFI) was determined by subtracting the respective background control from each sample for ATTO647.

FIG. 6A illustrate the effect of post-transfection time on cytometric resolution of Jurkat cells electroporated with RNP consisting of final concentrations of Cas9 protein were 1.5 or 0.15 μM and unlabeled crRNA and ATTO550-labeled tracrRNA. The ratio of gRNA:Cas9 protein:Alt-R® Cas9 Electroporation Enhancer was 1.2:1:1. Jurkat cells subjected to RNP but without electroporation were used as background controls. Gates were set to not include the background controls. Cells were sorted 24, 48 or 72 hours post-transfection. Prior to sorting, cells were washed 1 time with PBS+1% FBS. Histogram plots show fluorescence intensities for ATTO550.

FIG. 6B illustrates the positive cell fraction and mean fluorescence intensity of Jurkat cells were electroporated with RNP consisting of final concentrations of Cas9 protein were 1.5 or 0.15 μM and unlabeled crRNA and ATTO550-labeled tracrRNA as a function of post-transfection time. The experimental details are as described in FIG. 6A. The percentage of cells within the gate settings were determined and expressed as positive cell fraction. The mean fluorescence intensity (MFT) was determined by subtracting the respective background control from each sample for ATTO550.

FIG. 6C illustrate the effect of post-transfection time on cytometric resolution of Jurkat cells electroporated with RNP consisting of final concentrations of Cas9 protein were 1.5 or 0.15 μM and unlabeled crRNA and ATTO647-labeled tracrRNA. The ratio of gRNA:Cas9 protein:Alt-R® Cas9 Electroporation Enhancer was 1.2:1:1. Jurkat cells subjected to RNP but without electroporation were used as background controls. Gates were set to not include the background controls. Cells were sorted 24, 48 or 72 hours post-transfection. Prior to sorting, cells were washed 1 time with PBS +1% FBS. Histogram plots show fluorescence intensities for ATTO647.

FIG. 6D illustrates the positive cell fraction and mean fluorescence intensity of Jurkat cells were electroporated with RNP consisting of final concentrations of Cas9 protein were 1.5 or 0.15 μM and unlabeled crRNA and ATTO647-labeled tracrRNA as a function of post-transfection time. The experimental details are as described in FIG. 6C. The percentage of cells within the gate settings were determined and expressed as positive cell fraction. The mean fluorescence intensity (MFI) was determined by subtracting the respective background control from each sample for ATTO647.

FIG. 7A illustrates that the enrichment of sorted Jurkat cells leads to higher editing efficiencies under conditions of delivering 0.5 μM RNP to the cells by electroporation. Cells were electroporated with 1.5 μM or 0.5 μM RNP consisting of either unlabeled (Unlabeled) or labeled tracrRNA (ATTO550). The ratio of gRNA:Cas9 protein:Alt-R® Cas9 Electroporation Enhancer was 1.2:1:1. Jurkat cells subjected to RNP but without electroporation were used as background controls. Gates were set to not include the background controls. Cells were sorted 24 hours post-transfection and positive cells were re-plated and grown for an additional 48 hours. A population of the cells was not sorted, simply re-plated to serve as the unsorted control. After a 72-hour incubation, the genomic DNA was isolated, the target region amplified, digested with 2 U T7EI endonuclease, and the percent editing was determined by fragment separation on the Fragment Analyzer.

FIG. 7B illustrates enrichment of sorted HEK293 cells leads to identification of higher editing efficiencies under conditions of delivering 0.5 μM RNP to the cells by electroporation. Cells were electroporated with 1.5 μM or 0.5 μM RNP consisting of either unlabeled (Unlabeled) or labeled tracrRNA (ATTO550) as described in FIG. 7A.

FIG. 8A illustrates the detectability of fluorescently-labeled tracrRNA conjugated to an ATTO550-dye localized in cells by fluorescence microscopy when HEK293 cells stably expressing Cas9 were transfected using lipofection with unlabeled crRNA and ATTO550 labeled tracrRNA at a final concentration of 10 nM. (A) Detection of ATTO550-dye shows cellular localization.

FIG. 8B illustrates the an exemplary light microscopy image of the cells presented in FIG. 8A.

FIG. 9 illustrates the detection of fluorescently-labeled tracrRNA by fluorescence microscopy when HEK293 cells were electroporated using the Amaxa nucleofection (Lonza) with RNP made with unlabeled crRNA, and either ATTO488 or ATTO550 labeled tracrRNA, with a final concentration of 4 μM RNP. Electroporation was conducted in the presence of 4 Alt-R Electroporation Enhancer (IDT). Upper panels demonstrate detection of ATTO488-dye or ATTO550-dye which shows cellular localization. Lower panels demonstrate the light microscopic image of cells.

### DETAILED DESCRIPTION OF THE INVENTION

Labeling of components of the RNP allows for cellular visualization and fluorescent detection. For instance, a fusion protein comprised of Cas9 and GFP allows for cellular detection of the RNP. However, increasing the cargo size of the RNP can lead to lower delivery efficiency using lipofection due to incomplete complexation or the requirement of higher amounts of cationic lipids which can lead to cell toxicity and additional of large chimeric peptide domains to the Cas9 protein may affect the activity, cellular distribution, or halflife of the protein. The use of a super-negatively charged GFP can overcome some of these limitations (see Zuris et al., Nat Biotech (2015) 33(1):73-80). A more straightforward approach to detect the RNP is to label chemically synthesized guide RNA components with fluorescently labeled dyes, thereby avoiding unnecessary modification of the Cas9 protein.

A wide variety of reactive fluorescent reporter dyes are known in the literature. Typically, the fluorophore is an aromatic or heteroaromatic compound and can be a pyrene, anthracene, naphthalene, acridine, stilbene, indole, benzindole, oxazole, thiazole, benzothiazole, cyanine, carbocyanine, salicylate, anthranilate, coumarin, fluoroscein, rhodamine or other like compound. Suitable fluorescent reporters include xanthene dyes, such as fluorescein or rhodamine dyes, including 6-carboxyfluorescein (FAM), 2′7′-dimethoxy-4′S′-dichloro-6-carboxyfluorescein (JOE), tetrachlorofluorescein (TET), 6-carboxyrhodamine (R6G), N,N,N;N′-tetramethyl-6-carboxyrhodamine (TAMRA), 6-carboxy-X-rhodamine (ROX). Suitable fluorescent reporters also include the naphthylamine dyes that have an amino group in the alpha or beta position. For example, naphthylamino compounds include I-dimethylaminonaphthyl-S-sulfonate, 1-anilino-8-naphthalene sulfonate and 2-p-toluidinyl-6-naphthalene sulfonate, S-(2′-aminoethyl)aminonaphthalene-1-sulfonic acid (EDANS). Other fluorescent reporter dyes include coumarins, such as 3-phenyl-7-isocyanatocoumarin; acridines, such as 9-isothiocyanatoacridine and acridine orange; N-(p(2-benzoxazolyl)phenyl)maleimide; cyanines, such as indodicarbocyanine 3 (Cy3), indodicarbocyanine S (CyS), indodicarbocyanine S.S (CyS.S), 3-1-carboxy-pentyl)-3′-ethyl-S,S′-dimethyloxacarbocyanine (CyA); IH,SH,IIH, ISH-Xantheno[2,3,4-ij :S,6, 7 -i′j′]diquinolizin-18-ium, 9-[2(or 4)-[[[6-[2,S-dioxo-1-pyrrolidinyl)oxy ]-6-oxohexyl] amino ]sulfonyl]-4(or 2)-sulfophenyl]-2,3,6,7,12, 13, 16,17-octahydro-inner salt (TR or Texas Red); BODIPyrM dyes; benzoxaazoles; stilbenes; pyrenes; and the like.

While most of the fluorescent dyes used in molecular biology today have some water solubility, these water-soluble fluorescent dyes nevertheless are hydrophobic (lipophilic) and have the potential to interact strongly with lipid membranes (see Hughes et al., PlosOne (2014) 9(2):e87649). This characteristic can lead to potential problems during delivery of the RNP as it could prevent nuclear delivery of the RNP or the labeled guide RNA components, and thereby lead to inefficient genome editing. Furthermore, the fluorescently-labeled guide RNA components can bind non-specifically to the outside membranes of cells and result in false positives fluorescent imaging or cell sorting (when using fluorescence activated cell sorting (FACS)).

Several attachment chemistries are currently used for modifying oligonucleotides. For example, primary amino groups are widely used to attach modifiers, reporter moieties or labels to an oligonucleotide. In addition, they can be used to attach an oligonucleotide to a solid surface. Stable Schiff base linkers have been used for the synthesis of labeled oligonucleotides. (Dey & Sheppard (2001) Org. Lett. Vol. 3, 25:3983-3986, which is incorporated herein by reference). The methods have been limited to the post-synthetic attachment of labels, and the proposed methods have not been commercially viable alternatives to standard synthesis approaches. Previously described post-synthetic methods permit the incorporation of only a single type of reporter moiety or multiple copies of the same reporter moiety into an oligonucleotide.

Substituents can be attached to xanthene rings for bonding with various reagents, including oligonucleotides. For fluorescein and rhodamine dyes, appropriate linking methodologies for attachment to oligonucleotides have also been described. See for example, Khanna et al. U.S. Pat. No. 4,439,356; Marshall (1975) Histochemical J., 7:299-303; Menchen et al., U.S. Pat. No. 5,188,934; Menchen et al., European Patent Application No. 87310256.0; and Bergot et al., International Application PCT/U590/05565).

For purposes of this invention the terms, “linker” and “linking group” refer to a chemical group that is capable of reacting with a “complementary functionality” of a reagent. When the complementary functionality is an amine, preferred linking groups include such groups as isothiocyanate, sulfonylchloride, 4,6-dichlorotriazinyl, carboxylate, succinimidyl ester, other active carboxylate, e.g., —C(O)halogen, —C(O)OC₁₋₄alkyl, or —C(O)O C(O)C₁₋₄alkyl, amine, lower alkylcarboxy or —(CH₂)ₘN⁺(CH₃)₂(CH₂)ₘCOOH, wherein m is an integer ranging from 2 to 12. When the complementary functionality is a 5′-hydroxyl group of an oligonucleotide, the preferred linking group is a protected phosphoramidite. When the complementary functionality is sulfhydryl, the linking group can be a maleimide, halo acetyl, or iodoacetamide for example. See R. Haugland (1992) Molecular Probes Handbook of Fluorescent Probes and Research Chemicals, Molecular Probes, Inc., disclosing numerous modes for conjugating a variety of dyes to a variety of compounds which sections are incorporated herein by reference.

As used herein, the phrase “CRISPR ribonucleoprotein complex” refers to a ribonucleoprotein complex having CRISPR-associated endonuclease activity. Exemplary CRISPR ribonucleoprotein complexes include CRISPR/Cas9 CRISPR-associated endonuclease activity and CRISPR/Cpfl CRISPR-associated endonuclease activity.

As used herein, the phrase “CRISPR-associated RNA” refers to an RNA component that, when combined with a CRISPR-associated protein, results in an CRISPR ribonucleoprotein complex. Exemplary CRISPR ribonucleoprotein complexes include ribonucleoprotein complexes having an CRISPR-associated protein, such as CRISPR/Cas9 protein or CRISPR/Cpfl protein. An exemplary CRISPR-associated RNA includes a gRNA, including a crRNA and tracrRNA, for CRISPR/Cas9 protein that forms the CRISPR/Cas9 endonuclease system. Another exemplary CRISPR-associated RNA includes a crRNA for CRISPR/Cpfl protein that forms the CRISPR/Cpfl endonuclease system.

Examples of these CRISPR ribonucleoprotein complexes, the CRISPR-associated RNA and protein components, and CRISPR-associated endonuclease systems are disclosed in the following references: Collingwood, M. A., Jacobi, A. M., Rettig, G. R., Schubert, M. S., and Behlke, M. A., “CRISPR-BASED COMPOSITIONS AND METHOD OF USE,” U.S. patent application Ser. No. 14/975,709, filed Dec. 18, 2015, published now as U.S. Patent Application Publication No. US2016/0177304A1 on Jun. 23, 2016 and issued as U.S. Pat. No. 9,840,702 on Dec. 12, 2017; and Behlke, M. A. et al. “CRISPR/CPF1 SYSTEMS AND METHODS,” U.S. patent application Ser. No. 15/821736, filed Nov. 22, 2017, the contents of which are hereby incorporated by reference herein in their entirety.

The term “nucleic acid conjugate” refers to a nucleic acid conjugated to another molecule. The term “conjugated” and related verb tense phrases refer to covalent coupling (that is, via a chemical bond) of a first molecule to a second molecule. An exemplary nucleic acid conjugate, as described herein, includes a nucleic acid conjugated to a label that is configured to generate a signal. Exemplary labels configured to generate a signal include moieties having intrinsic fluorescence, chemiluminescence, phosphorescence or radioactive properties, enzymatic reactivity towards substrates having these properties, or having the ability to serve as a ligand for a binding partner having these properties.

A gRNA is comprised of a tracrRNA and crRNA. The crRNA and tracrRNA can be fused into a single chimeric nucleic acid (a single-guide RNA, or sgRNA) or they can be separate nucleic acids. The fluorescent dyes are placed on either the crRNA or the tracrRNA or both, or on the sgRNA. Neither location interferes with RNP delivery nor genome editing as editing efficiencies were comparable to non-labeled guide RNA components. In a further embodiment the label is placed on the crRNA. In another embodiment the label is placed on the tracrRNA. While placement on either crRNA or tracrRNA is feasible, the fluorescence signal and resolution is optimal when the label is on the tracrRNA. This is an unexpected finding as basic theory would suggest that dye labeling of the crRNA and the tracrRNA should perform equally well. The FACS resolution is dependent on the specific fluorescent dye used. If the fluorescent dye is placed on the crRNA a new dye-labeled crRNA must be manufactured for every different target needed. Alternatively, if the tracrRNA is labeled, the a single, universal dye-labeled tracrRNA can be manufactured which can be paired with any target-specific crRNA needed. Hence the dye-tracrRNA can be manufactured at large scale, lowering the cost of using this method. If a dye-labeled sgRNA is used, then a dye-labeled sgRNA must be manufactured for every target. Therefore, from a cost standpoint, use of a 2-part guide RNA complex comprising an unlabeled target-specific crRNA and a dye-labeled universal tracrRNA is preferred.

In one embodiment the label is a rhodamine-based dye or a zwitterionic dye. In another embodiment the label is a rhodamine-based dye, such as rhodamine 6G or rhodamine B dye. In another embodiment the label is ATTO550 dye, ATTO647 dye or ATTO488 dye (Atto-tech GmbH). Washing the cells after transfection helps to reduce the amount of non-specifically bound labeled tracrRNA, and the optimal sorting can be time-dependent. Fluorescence-activated cell sorting can be used to isolate the successfully transfected cell population, and thereby enrich for edited cells. The labeled tracrRNA can be visualized using fluorescence microscopy.

In another embodiment, a labeled crRNA is used in a Cpfl system. CRISPR/Cpfl is a DNA-editing technology analogous to the CRISPR/Cas9 system in that it is also an RNA-guided endonuclease of a class II CRISPR/Cas system (see Zetsche et al., Cell. (2015) 163(3):759-71). Since Cpfl is a smaller and simpler endonuclease than Cas9, its use can potentially overcome some of the limitations of the CRISPR/Cas9 system. While Cpfl was originally characterized from Prevotella and Francisella, many homologues of Cpfl exist from other bacterial species that have different properties. Codon optimized versions of the Cpfl enzymes from Acidaminococcus and Lachnospiraceae were shown to efficiently target DNMT1 in human cells, whereas the Prevotella and Francisella variants were inactive for genome editing in mammalian cells. In the present invention, the Cpfl crRNA can be labeled to detect transfection in the cell.

In one aspect, an isolated tracrRNA including a chemically-modified nucleotide or a non-nucleotide chemical modifier is provided. The isolated tracrRNA displays activity in the CRISPR-Cas endonuclease system. In one respect, the isolated tracrRNA includes a chemically- modified nucleotide having a modification selected from a group consisting of a ribose modification, an end-modifying group, and internucleotide modifying linkages. Exemplary ribose modifications include 2′O-alkyl (e.g., 2′OMe), 2′F, bicyclic nucleic acid, and locked nucleic acid (LNA). Exemplary end-modifying groups include a propanediol (C3) spacer and napthyl-azo modifier (N,N-diethyl-4-(4-nitronaphthalen-1-ylazo)-phenylamine, or “ZEN”), and an inverted-dT residue. Exemplary internucleotide modifying linkages include phosphorothioate modification.

In another aspect, an isolated crRNA including a chemically-modified nucleotide is provided. The isolated crRNA displays activity in the CRISPR-Cas endonuclease system. In one respect, the isolated crRNA includes a chemically-modified nucleotide having a modification selected from a group consisting of a ribose modification, an end modifying group, and internucleotide modifying linkage. Exemplary ribose modifications include 2′0-alkyl (e.g., 2′OMe), 2′F, bicyclic nucleic acid, and locked nucleic acid (LNA). Exemplary end-modifying groups include a propanediol (C3) spacer and napthyl-azo modifier (N,N-diethyl-4-(4-nitronaphthalen-1-ylazo)-phenylamine, or “ZEN”), and an inverted-dT residue. Exemplary internucleotide modifying linkages include phosphorothioate modification.

The terms “nucleic acid” and “oligonucleotide,” as used herein, refer to polydeoxyribonucleotides (containing 2-deoxy-D-ribose), polyribonucleotides (containing D-ribose), and to any other type of polynucleotide which is an N glycoside of a purine or pyrimidine base. There is no intended distinction in length between the terms “nucleic acid”, “oligonucleotide”, “oligomer” or “oligo”, and these terms will be used interchangeably. These terms refer only to the primary structure of the molecule. Thus, these terms include double- and single-stranded DNA, as well as double- and single-stranded RNA.

The terms “AS” and “antisense”, as used herein, refer to the complementary DNA strand opposite that of the strand that encodes the target gene. For example, “HPRT1 38285-AS” refers to a protospacer sequence located on the DNA strand opposite that of the human HPRT1 gene.

The term “population”, as used herein in reference to cells, generally refers to a group of cells that possess optical properties with respect to one or more measured parameters such that measured parameter data form a cluster in the data space. Populations of cells can be physically separated, based on those optical properties, via FACS.

The term “gate”, as used herein, generally refers to a boundary identifying a subset of data of interest. In cytometry, a gate may bound a group of events of particular interest. As used herein, “gating” generally refers to the process of defining a gate for a given set of data.

### Applications

In a first aspect, a method of detecting cells containing a CRISPR ribonucleoprotein complex is disclosed. The method includes a first step of contacting a CRISPR-associated RNA and a CRISPR-associated protein with a cell sample. The CRISPR-associated RNA is conjugated to a label configured to generate a signal. The CRISPR-associated protein is Cas9 protein or Cpfl protein. The method includes a second step of determining a presence of the signal from at least one cell in the cell sample. The presence of the signal from at least one cell in the cell sample detects at least one cell containing the CRISPR ribonucleoprotein complex. In a first respect, the method includes the additional step of enriching cells containing the CRISPR ribonucleoprotein complex from the cell sample using fluorescence-activated cell sorting. In a second respect, the methods provided above elaborate on the step of determining the presence of the signal from at least one cell in the cell sample comprises by using fluorescence microscopy or fluorescence-activated cell sorting. In a third respect, the methods provided above elaborate on the nature of the CRISPR-associated RNA. In one embodiment, the CRISPR-associated RNA is selected from a gRNA for Cas9 protein or a crRNA for the Cpfl protein. In another embodiment, the CRISPR-associated RNA is a gRNA for Cas9 protein, wherein the gRNA comprises a crRNA and a tracrRNA, further wherein the tracrRNA is conjugated to a label comprising a fluorescent dye. In yet another embodiment, the CRISPR-associated RNA is a crRNA for the Cpfl protein, further wherein the crRNA is conjugated to a label comprising a fluorescent dye. In a fourth respect, the tracrRNA is conjugated to a label that includes a fluorescent dye. In a preferred embodiment of the fourth respect, the fluorescent dye is hydrophilic. In a preferred embodiment of the fourth respect, the fluorescent dye is a rhodamine- based dye or a zwitterionic dye. In another embodiment, the label is a rhodamine-based dye, such as rhodamine 6G or rhodamine B dye. In a preferred embodiment of the fourth respect, the fluorescent dye is ATTO550 dye, ATTO647 dye or ATTO488 dye.

In a second aspect, a nucleic acid conjugate comprising an RNA conjugated to a label configured to generate a signal is provided. The RNA is selected from the group consisting of a gRNA, a crRNA and a tracrRNA. In a first respect, the CRISPR-associated RNA is conjugated to a label comprising a fluorescent dye. In one preferred embodiment of the first respect, the fluorescent dye is hydrophilic. In a preferred embodiment of the first respect, the fluorescent dye is a zwitterionic dye or a rhodamine-based dye, such as, for example, rhodamine 6G or rhodamine B. In a preferred embodiment of the first respect, the fluorescent dye is ATTO550 dye, ATTO647 dye or ATTO488 dye.

In a third aspect, a kit for detecting cells containing a CRISPR ribonucleoprotein complex is provided. In preferred embodiments, the kit comprising a nucleic acid conjugate according to any of respects set forth in the second aspect described above. In an additional embodiment of the third aspect, the kit includes a positive cell control. The positive cell control includes a CRISPR ribonucleoprotein complex comprising a Cas9 protein or a Cpfl protein and a nucleic acid conjugate according to any of respects set forth in the second aspect described above. In an additional embodiment of the third aspect and first respect thereof set forth above, the kit includes a negative cell control. The negative cell control includes a CRISPR ribonucleoprotein complex comprising a Cas9 protein or a Cpfl protein and an unlabeled CRISPR-associated RNA is selected from the group consisting of a crRNA and a tracrRNA.

The following examples further illustrate the invention but, of course, should not be construed as in any way limiting its scope.

### EXAMPLE 1

This example demonstrates whether coupling of fluorescent dyes onto the crRNA or tracrRNA affects the efficiency of the genome editing.

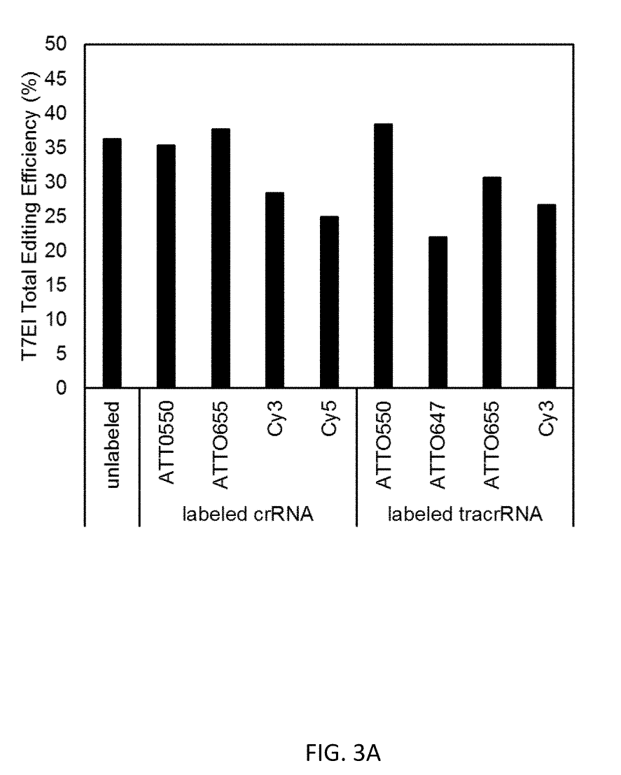

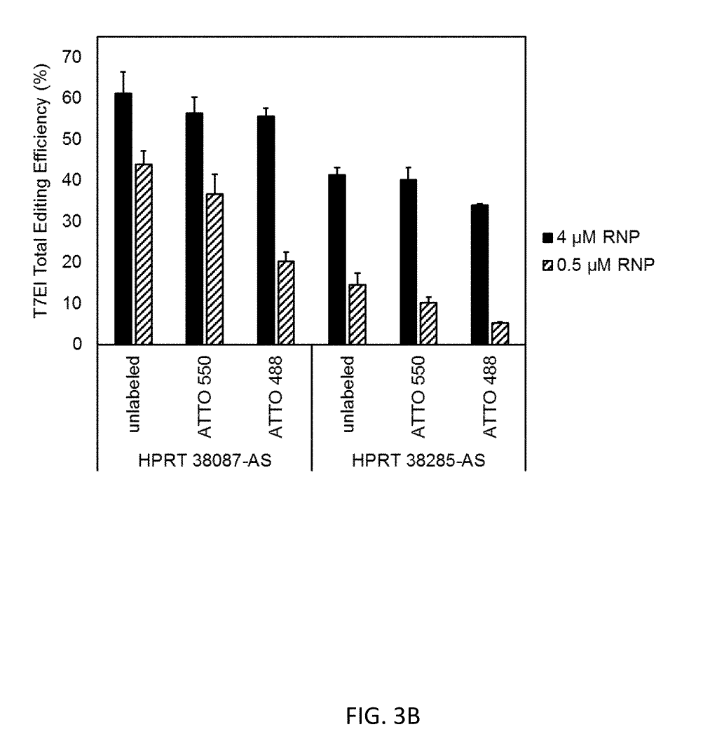

It was hypothesized that the coupling of a dye to crRNA or tracrRNA could alter the structure of the guide RNA complex, and therefore can influence the interaction with Cas9 protein. To test this, guide RNA complexes were generated that have either a labeled crRNA or a labeled tracrRNA. These guide RNAs were complexed to Cas9 protein (SEQ ID NO:1) to form the ribonucleoprotein complex (RNP), and were then transfected into HEK293 cells using cationic lipids or electroporation. Editing efficiencies were determined using a T7 Endonuclease I (T7EI) assay. FIG. 3 shows that editing efficiencies are not affected significantly by placing fluorescent dyes on either the crRNA or the tracrRNA molecule compared to the unlabeled guide RNA control.

For lipofection experiments, HEK293 cells were transfected using RNAiMAX (Thermo Fisher). The guide RNA complex was formed by hybridization of equal molar amounts of crRNA and tracrRNA at a final concentration of 100 μM in IDT Duplex Buffer (30 mM HEPES, pH 7.5, 100 mM Potassium Acetate). The crRNA was specific to the HPRT1 gene at position 38285-AS. Either unlabeled crRNA (SEQ ID NO:2), ATTO550-labeled crRNA (SEQ ID NO:7), ATTO655-labeled crRNA (SEQ ID NO:9), Cy3-labeled crRNA (SEQ ID NO:10), or Cy5-labeled crRNA (SEQ ID NO:11) was used. Either unlabeled tracrRNA (SEQ ID NO:3), ATTO55-labeled tracrRNA (SEQ ID NO:13), ATTO647-labeled tracrRNA (SEQ ID NO:14), ATTO655-labeled tracrRNA (SEQ ID NO:15), or Cy3-labeled tracrRNA (SEQ ID NO:16) was used. The ribonucleoprotein complex (RNP) was generated by complexation of 1.5 pmol Cas9 protein with 1.5 pmol guide RNA complex in OptiMEM (Invitrogen) in a total volume of 50 μL. The RNP complex was then added to 4E4 HEK293 cells diluted in 100 μL growth media. Genomic DNA was isolated after the cells were incubated for 48 hours at 37° C. containing 5% CO₂. The targeted genomic locus was amplified using PCR (SEQ ID Nos. 5,6) Heteroduplexes were formed by denaturing the amplicons followed by a slow cool-down. Mismatches in heteroduplexes were cleaved by 2 U T7 Endonuclease I, and cleaved and non-cleaved products were quantified using a Fragment Analyzer.

For electroporation experiments, HEK293 cells were electroporated using the Amaxa Nucleofector System (Lonza). After harvesting the cells using trypsinization and subsequent neutralization of the trypsin by addition of growth media containing 10% Fetal Bovine Serum (FBS), cells were counted and pelleted using centrifugation (200 rpm, 10 minutes at room temperature). The pelleted cells were washed with one volume of at least 5 mL 1× phosphate-buffered saline (PBS). The cells were then pelleted and resuspended in Nucleofection Solution SF at a concentration of 1.8E7 cells/mL. The guide RNA complex was formed by hybridization of equal molar amounts of crRNA and tracrRNA at a final concentration of 30 μM in IDTE. The unlabeled crRNA was specific to the HPRT 1 gene at position 38285-AS (SEQ ID NO:2) or at position 38087-AS (SEQ ID NO:12). Either unlabeled tracrRNA (SEQ ID NO:3), ATTO550-labeled tracrRNA (SEQ ID No.13), or ATTO-488-labeled tracrRNA (SEQ ID NO:17) was used. The ribonucleoprotein complex (RNP) was generated by complexation of 201 pmol Cas9 protein with 201 pmol guide RNA complex in a total volume of 10 μL. 1× PBS was used to adjust to the final volume. Following mixing, complexes were formed by incubation of the RNP for 10-20 minutes at room temperature. For each electroporation, 6 μL of RNP complex was added to 20 μL of HEK293 cells in Nucleofection Solution SF (3.5E5 cells). Additionally, 4μL of Alt-R® Cas9 Electroporation Enhancer, diluted in IDTE, was added to achieve a final concentration of 4 μM. 25 μL out of 30 μL of the solution was mixed by pipetting up and down and transferred to an electroporation cuvette. The cells were electroporated according to the manufacturer's protocol using the Amaxa 96-well Shuttle device and nucleofection settings 96-DS-150. After electroporation, the cells were resuspended with 75 μL pre-warmed culture media in the electroporation cuvette. Triplicate aliquots of 25 μL of resuspended cells were further cultured in 175 μL pre-warmed media each. Genomic DNA was isolated after the cells were incubated for 48 hours at 37° C. containing 5% CO₂. The targeted genomic locus was amplified using PCR (SEQ ID Nos. 5,6). Heteroduplexes were formed by denaturing the amplicons followed by a slow cool-down. Mismatches in heteroduplexes were cleaved by 2.5 U T7 Endonuclease I, and cleaved and non-cleaved products were quantified using a Fragment Analyzer. Percent cleavage of targeted DNA was calculated as the average molar concentration of the cut products/(average molar concentration of the cut products +molar concentration of the uncut band)×100.

WT SpyCas9 AA sequence with added NLS domains and
a HIS-Tag purification domain (in bold).
SEQ ID NO: 1
MGSSAPKKKRKVGIHGVPAAMDKKYSIGLDIGTNSVGWAVITDEYKVPSK
KFKVLGNTDRHSIKKNLIGALLFDSGETAEATRLKRTARRRYTRRKNRIC
YLQEIFSNEMAKVDDSFFHRLEESFLVEEDKKHERHPIFGNIVDEVAYHE
KYPTIYHLRKKLVDSTDKADLRLIYLALAHMIKFRGHFLIEGDLNPDNSD
VDKLFIQLVQTYNQLFEENPINASGVDAKAILSARLSKSRRLENLIAQLP
GEKKNGLFGNLIALSLGLTPNFKSNFDLAEDAKLQLSKDTYDDDLDNLLA
QIGDQYADLFLAAKNLSDAILLSDILRVNTEITKAPLSASMIKRYDEHHQ
DLTLLKALVRQQLPEKYKEIFFDQSKNGYAGYIDGGASQEEFYKFIKPIL
EKMDGTEELLVKLNREDLLRKQRTFDNGSIPHQIHLGELHAILRRQEDFY
PFLKDNREKIEKILTFRIPYYVGPLARGNSRFAWMTRKSEETITPWNFEE
VVDKGASAQSFIERMTNFDKNLPNEKVLPKHSLLYEYFTVYNELTKVKYV
TEGMRKPAFLSGEQKKAIVDLLFKTNRKVTVKQLKEDYFKKIECFDSVEI
SGVEDRFNASLGTYHDLLKIIKDKDFLDNEENEDILEDIVLTLTLFEDRE
MIEERLKTYAHLFDDKVMKQLKRRRYTGWGRLSRKLINGIRDKQSGKTIL
DFLKSDGFANRNFMQLIHDDSLTFKEDIQKAQVSGQGDSLHEHIANLAGS
PAIKKGILQTVKVVDELVKVMGRHKPENIVIEMARENQTTQKGQKNSRER
MKRIEEGIKELGSQILKEHPVENTQLQNEKLYLYYLQNGRDMYVDQELDI
NRLSDYDVDHIVPQSFLKDDSIDNKVLTRSDKNRGKSDNVPSEEVVKKMK
NYWRQLLNAKLITQRKEDNLTKAERGGLSELDKAGFIKRQLVETRQITKH
VAQILDSRMNTKYDENDKLIREVKVITLKSKLVSDFRKDFQFYKVREINN
YHHAHDAYLNAVVGTALIKKYPKLESEFVYGDYKVYDVRKMIAKSEQEIG
KATAKYFFYSNIMNFFKTEITLANGEIRKRPLIETNGETGEIVWDKGRDF
ATVRKVLSMPQVNIVKKTEVQTGGFSKESILPKRNSDKLIARKKDWDPKK
YGGFDSPTVAYSVLVVAKVEKGKSKKLKSVKELLGITIMERSSFEKNPID
FLEAKGYKEVKKDLIIKLPKYSLFELENGRKRMLASAGELQKGNELALPS
KYVNFLYLASHYEKLKGSPEDNEQKQLFVEQHKHYLDEIIEQISEFSKRV
ILADANLDKVLSAYNKHRDKPIREQAENIIHLFTLTNLGAPAAFKYFDTT
IDRKRYTSTKEVLDATLIHQSITGLYETRIDLSQLGGDAAPKERRKVDPK
ERRKVAAALEHHHHHH

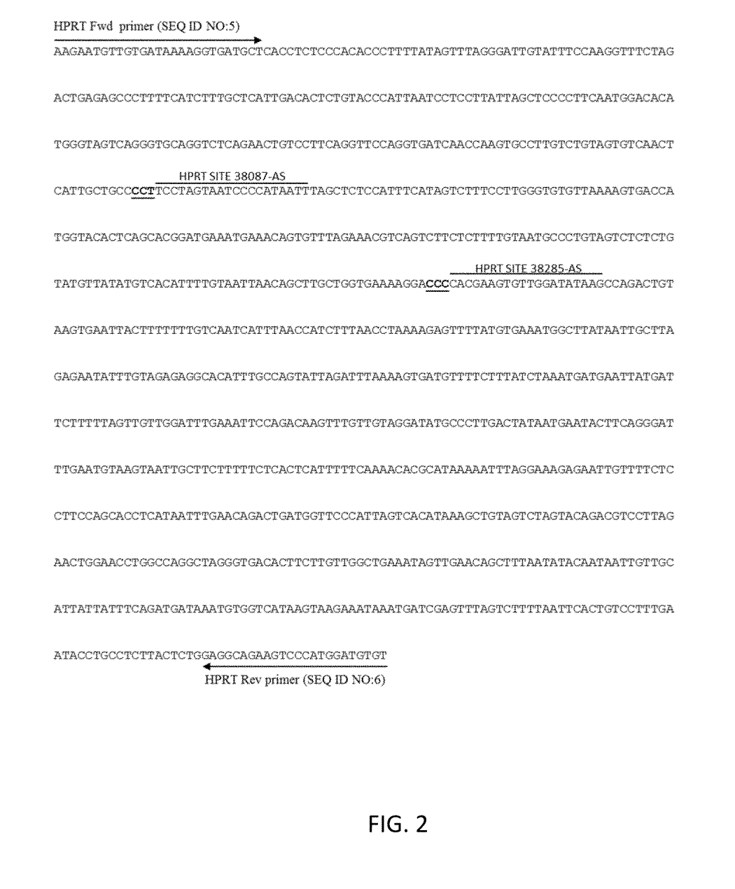

TABLE 1
Sequences of PCR primers used to amplify HPRT1
target after editing, as shown in FIG. 2, in
order for the gene editing efficiency to
be determined via the T7EI assay.

| | | SEQ |
|---|---|---|
| Primer Name | Primer Sequence | ID NO: |
| HPRT1 forward | AAGAATGTTGTGATAAAAGGTGATGCT | 5 |
| HPRT1 reverse | ACACATCCATGGGACTTCTGCCTC | 6 |

TABLE 2
Sequences of crRNA components

| crRNA Name | crRNA sequence | SEQ ID NO: |
|---|---|---|
| HPRT1 38285-AS | /5SpC3/rCrUrUrArUrArUrCrCrArArCrArCrUrU | 2 |
| crRNA Unlabeled | rCrGrUrGrGrUrUrUrUrArGrArGrCrUrArUrGrCr | |
| | U/3SpC3/ | |
| HPRT1 38285-A5 | /5SpC3/rCrUrUrArUrArUrCrCrArArCrArCrUrU | 7 |
| crRNA ATTO550 | rCrGrUrGrGrUrUrUrUrArGrArGrCrUrArUrGrCr | |
| | U/3A110550N/ | |
| HPRT1 38285-A5 | /5SpC3/rCrUrUrArUrArUrCrCrArArCrArCrUrU | 8 |
| crRNA ATTO647 | rCrGrUrGrGrUrUrUrUrArGrArGrCrUrArUrGrCr | |
| | U/3A110647N/ | |
| HPRT1 38285-AS | /5SpC3/rCrUrUrArUrArUrCrCrArArCrArCrUrU | 9 |
| crRNA ATTO655 | rCrGrUrGrGrUrUrUrUrArGrArGrCrUrArUrGrCr | |
| | U/3ATTO655N/ | |
| HPRT1 38285-AS | /5SpC3/rCrUrUrArUrArUrCrCrArArCrArCrUrU | 10 |
| crRNA Cy3 | rCrGrUrGrGrUrUrUrUrArGrArGrCrUrArUrGrCr | |
| | U/3Cy3N/ | |
| HPRT1 38285-AS | /5SpC3/rCrUrUrArUrArUrCrCrArArCrArCrUrU | 11 |
| crRNA Cy5 | rCrGrUrGrGrUrUrUrUrArGrArGrCrUrArUrGrCr | |
| | U/3Cy5N/ | |
| HPRT1 38087-AS | /5SpC3/rArArUrUrArUrGrGrGrGrArUrUrArCrU | 12 |
| crRNA Unlabeled | rArGrGrArGrUrUrUrUrArGrArGrCrUrArUrGrCr | |
| | U/3SpC3/ | |

Abbreviations: rA, rG, rC, rU = RNA bases; mA, mC, mG, mU = 2′OMe RNA bases;
“*” = phosphorothioate internucleotide modifications; /5SpC3/ = 5′ C3 spacer;
3SpC3/ = 3′ C3 spacer; /3Cy5N/ = 3′ Cy5, coupled as NHS-ester dye to amino-mod
RNA;
/3Cy3_N/ = 3′ Cy3, coupled as NHS-ester dye to amino-mod RNA;
/3ATTO550N/ = 3′ ATTO550, coupled as NHS-ester dye to amino-mod RNA;
/3ATTO647N/ = 3′ ATTO647, coupled as NHS-ester dye to amino-mod RNA; and
/3ATTO655N/ = 3′ ATTO655, coupled as NHS-ester dye to amino-mod RNA.

TABLE 3
Sequences of tracrRNA components

| tracrRNA Name | tracrRNA sequence | SEQ ID NO: |
|---|---|---|
| tracrRNA | mA*mG*mCmAmUmAmGmCmArArGrUrUrArArArArUrArArGrG | 3 |
| Unlabeled | rCrUrArGrUrCrCrGrUrUrArUrCrArAmCmUmUmGmAmAmAmA | |
| | mAmGmUmGmGmCmAmCmCmGmAmGmUmCmGmGmUmGmCmU*mU*mU | |
| tracrRNA | /5ATTO550N/mA*mG*mCmAmUmAmGmCmArArGrUrUrArArAr | 13 |
| ATPO550 | ArUrArArGrGrCrUrArGrUrCrCrGrUrUrArUrCrArAmCmUm | |
| | UmGmAmAmAmAmAmGmUmGmGmCmAmCmCmGmAmGmUmCmGmGmUm | |
| | GmCmU*mU*mU | |
| tracrRNA | /5ATTO647N/mA*mG*mCmAmUmAmGmCmArArGrUrUrArArAr | 14 |
| ATTO647 | ArUrArArGrGrCrUrArGrUrCrCrGrUrUrArUrCrArAmCmUm | |
| | UmGmAmAmAmAmAmGmUmGmGmCmAmCmCmGmAmGmUmCmGmGmUm | |
| | GmCmU*mU*mU | |
| tracrRNA | /5ATTO655N/mA*mG*mCmAmUmAmGmCmArArGrUrUrArArAr | 15 |
| ATTO655 | ArUrArArGrGrCrUrArGrUrCrCrGrUrUrArUrCrArAmCmUm | |
| | UmGmAmAmAmAmAmGmUmGmGmCmAmCmCmGmAmGmUmCmGmGmUm | |
| | GmCmU*mU*mU | |
| tracrRNA | /5Cy3/mA*mG*mCmAmUmAmGmCmArArGrUrUrArArArArUrA | 16 |
| Cy3 | rArGrGrCrUrArGrUrCrCrGrUrUrArUrCrArAmCmUmUmGmA | |
| | mAmAmAmAmGmUmGmGmCmAmCmCmGmAmGmUmCmGmGmUmGmCmU | |
| | *mU*mU | |
| tracrRNA | /5ATTO588N/mA*mG*mCmAmUmAmGmCmArArGrUrUrArArAr | 17 |
| ATTO488 | ArUrArArGrGrCrUrArGrUrCrCrGrUrUrArUrCrArAmCmUm | |
| | UmGmAmAmAmAmAmGmUmGmGmCmAmCmCmGmAmGmUmCmGmGmUm | |
| | GmCmU*mU*mU | |

Abbreviations:
rA, rG, rC, rU = RNA bases; mA, mC, mG, mU = 2′OMe RNA bases;
“*” = phosphorothioate internucleotide modification; /5SpC3/ = 5′ C3 spacer;
/3Cy5N/ = 3′ Cy5, coupled as NETS-ester dye to amino-mod RNA;
/3 Cy3 N/ = 3′ Cy3, coupled as NHS-ester dye to amino-mod RNA;
/5ATTO550N/ = 5′ ATTO550, coupled as NETS-ester dye to amino-mod RNA;
/5ATTO647N/ = 5′ ATTO647, coupled as NETS-ester dye to amino-mod RNA;
5ATTO655N/ = 5′ ATTO655, coupled as NHS-ester dye to amino-mod RNA
SEQ ID NO: 4
1083 bp region (bases 39387-40469 of Genbank entry; ID: M26434.1) of the human
hypoxanthine phosphoribosyl transferase (HPRT1) gene used herein as a target
tp access and compare the gene editing efficiency of CRISPR systems with the
various constructs disclosed within.
AAGAATGTTGTGATAAAAGGTGATGCTCACCTCTCCCACACCCTTTTATAGTTTAGGGATTGTATTTCCAAGGTTTC
TAGACTGAGAGCCCTTTTCATCTTTGCTCATTGACACTCTGTACCCATTAATCCTCCTTATTAGCTCCCCTTCAATG
GACACATGGGTAGTCAGGGTGCAGGTCTCAGAACTGTCCTTCAGGTTCCAGGTGATCAACCAAGTGCCTTGTCTGTA
GTGTCAACTCATTGCTGCCCCTTCCTAGTAATCCCCATAATTTAGCTCTCCATTTCATAGTCTTTCCTTGGGTGTGT
TAAAAGTGACCATGGTACACTCAGCACGGATGAAATGAAACAGTGTTTAGAAACGTCAGTCTTCTCTTTTGTAATGC
CCTGTAGTCTCTCTGTATGTTATATGTCACATTTTGTAATTAACAGCTTGCTGGTGAAAAGGACCCCACGAAGTGTT
GGATATAAGCCAGACTGTAAGTGAATTACTTTTTTTGTCAATCATTTAACCATCTTTAACCTAAAAGAGTTTTATGT
GAAATGGCTTATAATTGCTTAGAGAATATTTGTAGAGAGGCACATTTGCCAGTATTAGATTTAAAAGTGATGTTTTC
TTTATCTAAATGATGAATTATGATTCTTTTTAGTTGTTGGATTTGAAATTCCAGACAAGTTTGTTGTAGGATATGCC
CTTGACTATAATGAATACTTCAGGGATTTGAATGTAAGTAATTGCTTCTTTTTCTCACTCATTTTTCAAAACACGCA
TAAAAATTTAGGAAAGAGAATTGTTTTCTCCTTCCAGCACCTCATAATTTGAACAGACTGATGGTTCCCATTAGTCA
CATAAAGCTGTAGTCTAGTACAGACGTCCTTAGAACTGGAACCTGGCCAGGCTAGGGTGACACTTCTTGTTGGCTGA
AATAGTTGAACAGCTTTAATATACAATAATTGTTGCATTATTATTTCAGATGATAAATGTGGTCATAAGTAAGAAAT
AAATGATCGAGTTTAGTCTTTTAATTCACTGTCCTTTGAATACCTGCCTCTTACTCTGGAGGCAGAAGTCCCATGGA
TGTGT

### EXAMPLE 2

The following example demonstrates the level of non-specific binding of labeled components by comparing electroporated vs non-electroporated cells both in the presence of RNPs containing labeled crRNA or tracrRNA.

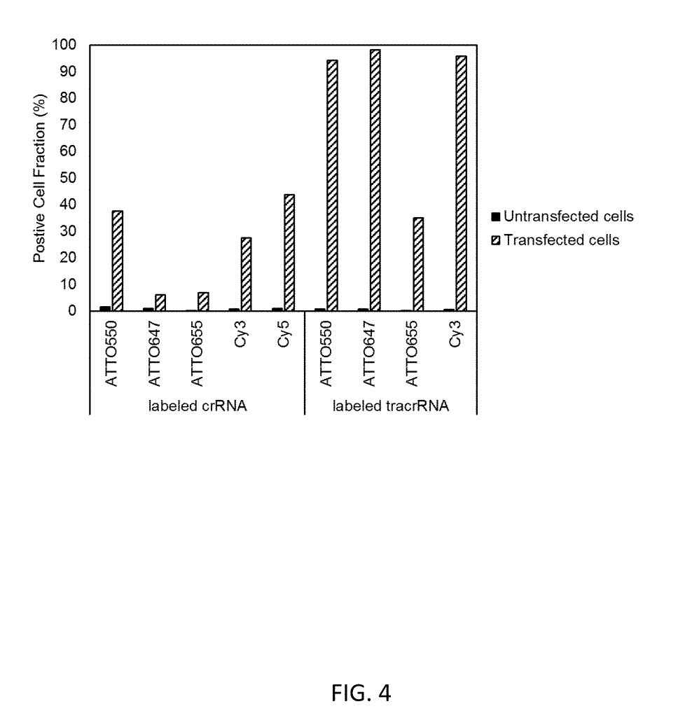

Fluorescent dyes can display strong interactions with a lipid membrane (see Hughes). As such, the fluorescently-labeled crRNAs or tracrRNAs could potentially bind to cells during transfection without being internalized, and would then lead to false positives when sorting positively labeled cells. The level of non-specific binding was determined by comparing electroporated vs non-electroporated cells both in the presence of RNPs containing labeled crRNA or tracrRNA. Gates were set using the non-electroporated controls such that fluorescently-labeled cells in the non-electroporated conditions were not selected. FIG. 4 shows the percentage of cells that are above (non-electroporated) background levels. Overall, the percentage of positively sorted cells using labeled crRNA is relatively low compared to labeled tracrRNA with the exception of ATTO655-tracrRNA. This indicates that the resolution of positively sorted cells using labeled crRNA under these conditions is lower, and leads to lower yields. Cells labeled with ATTO550-tracrRNA, ATTO647-tracrRNA, or Cy3-tracrRNA can be resolved more efficiently (>90%).

Jurkat cells were electroporated using the Neon Transfection System (Invitrogen). Cells were counted and pelleted using centrifugation (600 rpm, 10 minutes at room temperature). The pelleted cells were washed with one volume of at least 5 mL 1× phosphate-buffered saline (PBS). The cells were then pelleted and resuspended in Buffer R at a concentration of 1.11E7 cells/mL. The guide RNA complex was formed by hybridization of equal molar amounts of crRNA and tracrRNA at a final concentration of 45 μM in IDTE. The crRNA was specific to the HPRT1 gene at position 38285-AS. Either unlabeled crRNA (SEQ ID NO:2), ATTO550-labeled crRNA (SEQ ID NO:7), ATTO647-labeled crRNA (SEQ ID NO:8), ATTO655-labeled crRNA (SEQ ID NO:9), Cy3-labeled crRNA (SEQ ID NO:10), or Cy5-labeled crRNA (SEQ ID NO:11) was used. Either unlabeled tracrRNA (SEQ ID NO:3), ATTO550-labeled tracrRNA (SEQ ID NO:13), ATTO647-labeled tracrRNA (SEQ ID NO:14), ATTO655-labeled tracrRNA (SEQ ID NO:15), or Cy3-labeled tracrRNA (SEQ ID NO:16) was used. The ribonucleoprotein complex (RNP) was generated by complexation of 150 pmol Cas9 protein with 180 pmol guide RNA complex in a total volume of 10 μL. Buffer R was used to adjust to the final volume. Following mixing, complexes were formed by incubation of the RNP for 10-20 minutes at room temperature. For each electroporation, 10 μL of RNP complex was added to 90 μL of Jurkat cells in Buffer R (1E6 cells). Final Cas9 concentration was 1.5 μM. Final guide RNA concentration was 1.8 μM. Additionally, 20 μL of Alt-R® Cas9 Electroporation Enhancer, diluted in IDTE, was added to achieve its desired final concentration of 1.8 μM. 100 μL out of 120 μL of the solution was mixed by pipetting up and down and transferred to a Neon electroporation tip. The cells were electroporated using the following parameters: 1600 V, 10 ms, 3 pulses. After electroporation, the cells were added to 1 mL of pre-warmed culture media in 12-well culture plates. After 24-hour incubation, the cells were spun down, washed with FACS Buffer (1× PBS +1% FBS+0.1% Sodium Azide), spun down again, and resuspended in FACS Buffer before FACS analysis. Cells were sorted on a BD LSR II (BD Biosciences) using methods well known to those with ordinary skill in the art.

### EXAMPLE 3

The following example outlines the optimal cell preparation methods for cytometric analysis, and an example of multiple post-transfection times used to generate signal.

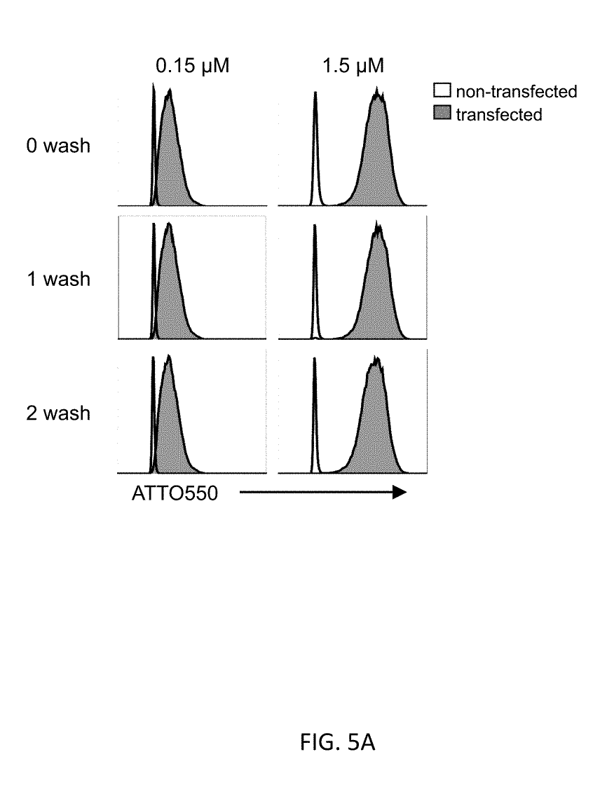

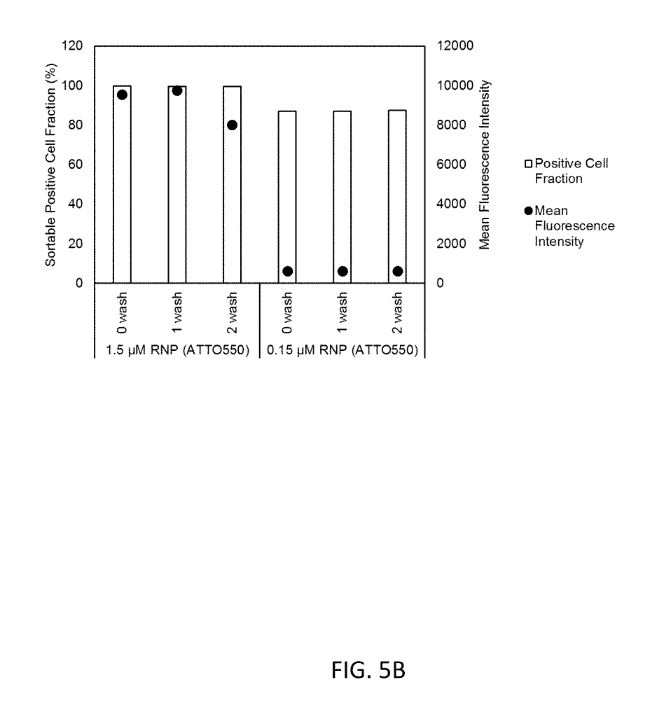

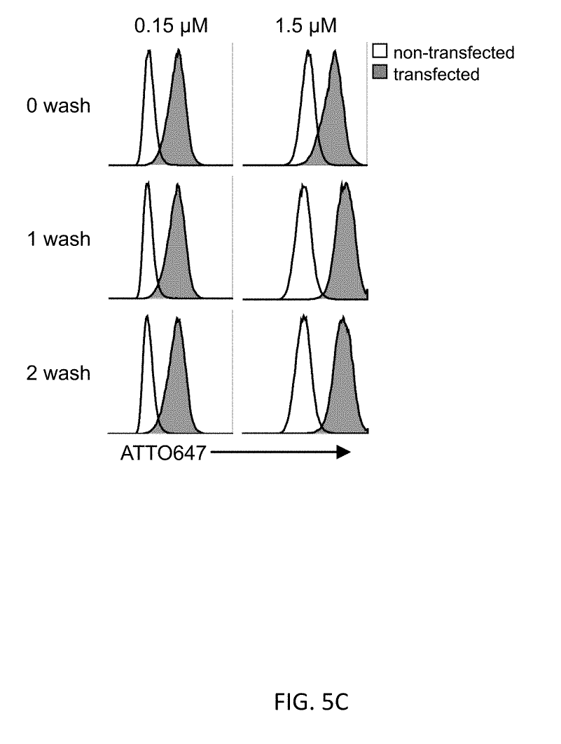

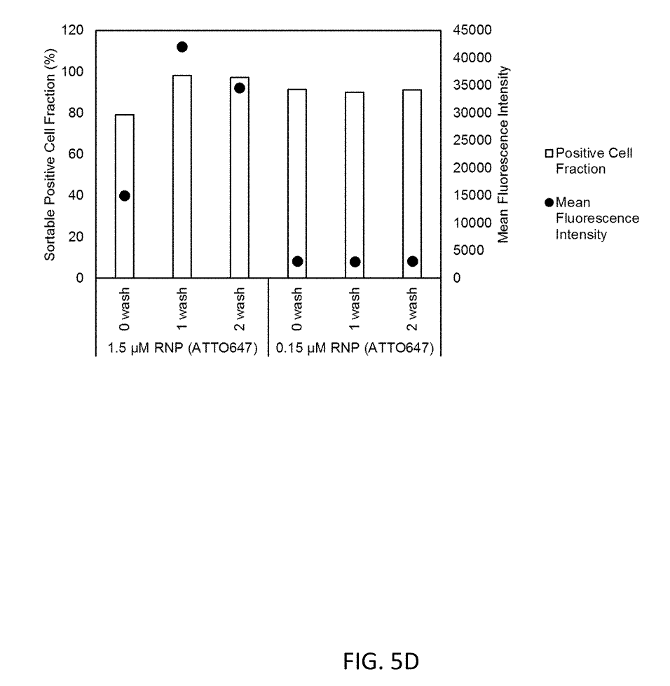

Example 2 showed that limiting non-specific binding of fluorescently-labeled guide RNA components can be achieved by selection of the right fluorescent dye as well as placing this dye on the tracrRNA (FIG. 4). Washing the cells before cytometric analysis decreases the non- specific binding further (FIG. 5). However, this benefit is dependent on the fluorescent dye used, as well as the concentration of the RNP. We compared 0, 1, and 2 wash steps prior to cytometric analysis of Jurkat cells. Additionally, we tested two RNP concentrations (0.15 μM and 1.5 μM), and two fluorescent dyes (ATTO550 and ATTO647). FIG. 5A shows that for ATTO550, at different RNP concentrations, the transfected cells show much brighter fluorescent intensities at all washing steps compared to the non-electroporated controls. This resolution allows for high levels of positively sortable cells (FIG. 5B). Furthermore, there is a positive correlation between RNP concentration and mean fluorescent intensity (MFI), which allows for successful sorting (FIG. 5B). The effects of washing prior to cytometric analysis is clearer when ATTO647 is used as fluorescent dye at the higher RNP concentration (FIG. 5C). With subsequent washes, the transfected cells show less overlap with non-transfected cells, indicating a reduced amount of non-specific binding. As a result, the number of positive (sortable) cells is increased (FIG. 5D). Thus, washing cells prior to sorting can lead to increased amounts of positive cells, and therefore can lead to higher editing efficiencies, but is dye-dependent. Similar results were seen with HEK293 cells (data not shown).

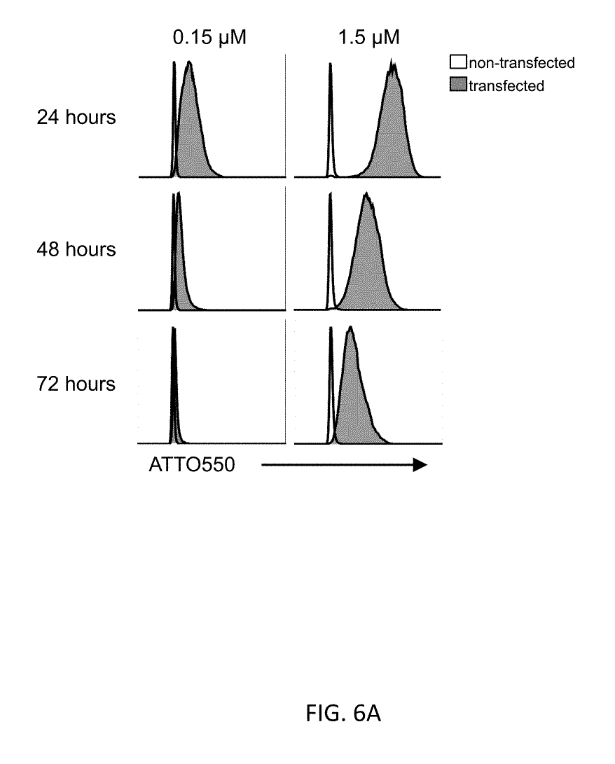

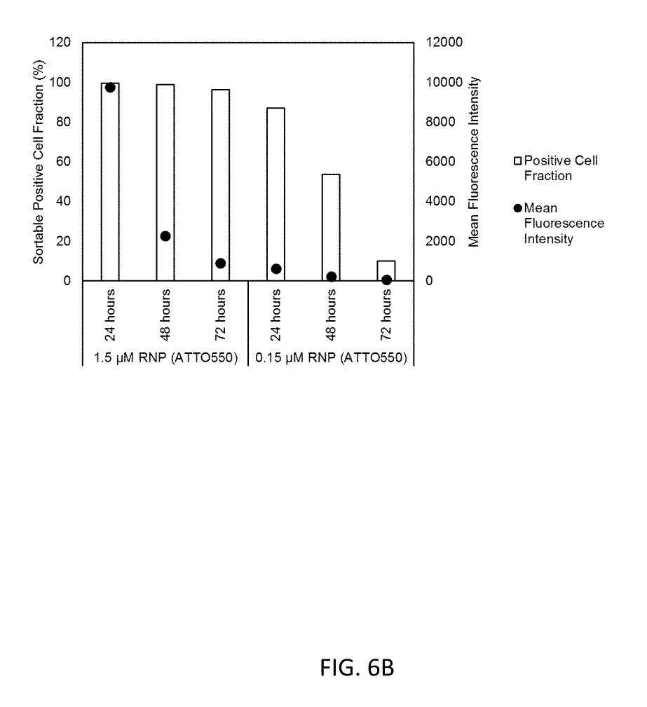

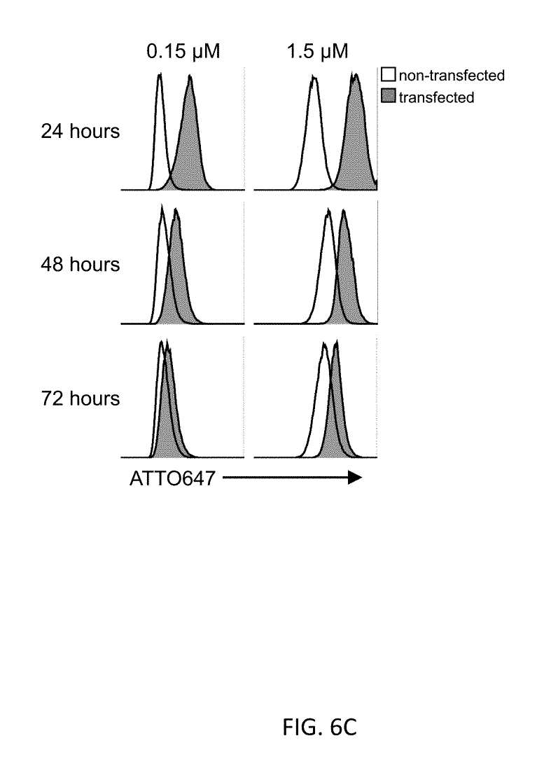

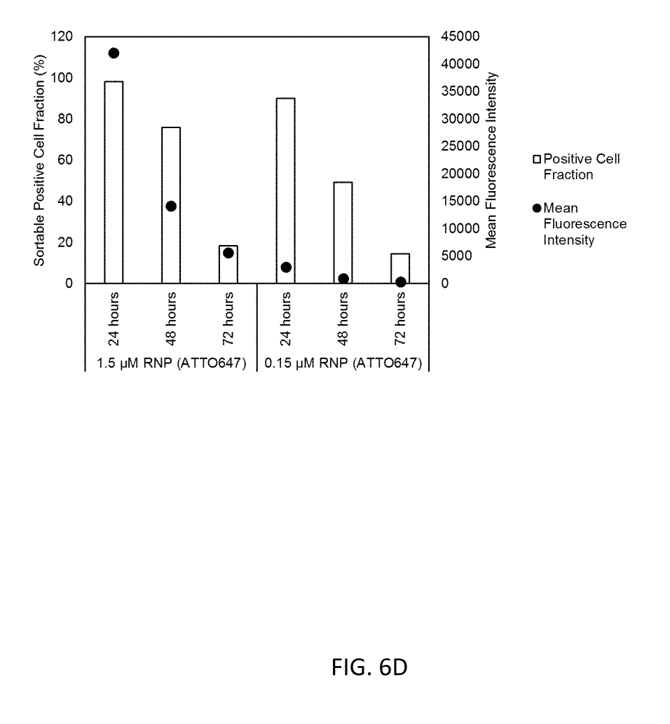

Fluorescence levels of transfected vs non-transfected Jurkat cells were measured 24, 48, and 72 hours after transfection using two different RNP concentrations (0.15 μM and 1.5 μM) and two fluorescent dyes (ATTO550 and ATTO647). For ATTO550, the fluorescence intensity was brighter at a RNP concentration of 1.5 μM compared to 0.15 μM (FIG. 6A) for each time point. For both RNP concentrations, the fluorescent intensity decreases with time. This results in a decrease of positive (sortable) cells, which is correlated to the mean fluorescent intensity (FIG. 6B). The same effects are seen when ATTO647 is used as fluorescent dye (FIGS. 6 C, D). Therefore, we show that sorting cells at 24 hours post-transfection leads to optimal sortability of cells, independent of fluorescent dye used. Extended times, however, were possible. Similar results were seen with HEK293 cells (data not shown).

Jurkat cells were transfected using the Neon Transfection system (1E6 cells/1600V/10 ms/3pulses) targeting the HPRT1 gene at position 38285-AS. Guide RNA complexes were made with unlabeled crRNA (SEQ ID NO:2) and ATTO 550-labeled tracrRNA (SEQ ID NO:13) or ATTO647-labeled tracrRNA (SEQ ID NO:14). RNP concentrations of 1.5 and 0.15 μM were used. After electroporation, the cells were added to 1 mL of pre-warmed culture media in 12-well culture plates. After a 24-, 48-, or 72-hour incubation, the cells were spun down. The cells were either not washed, or washed once or twice with FACS Buffer, then spun down again. Cells were resuspended in FACS Buffer before FACS analysis. Cells were sorted on a BD LSR II (BD Biosciences) using methods well known to those with ordinary skill in the art.

### EXAMPLE 4

The following example demonstrates that enriching the cell sample through FACS sorting of RNP-containing cells leads to better editing efficiency of the resulting sample.

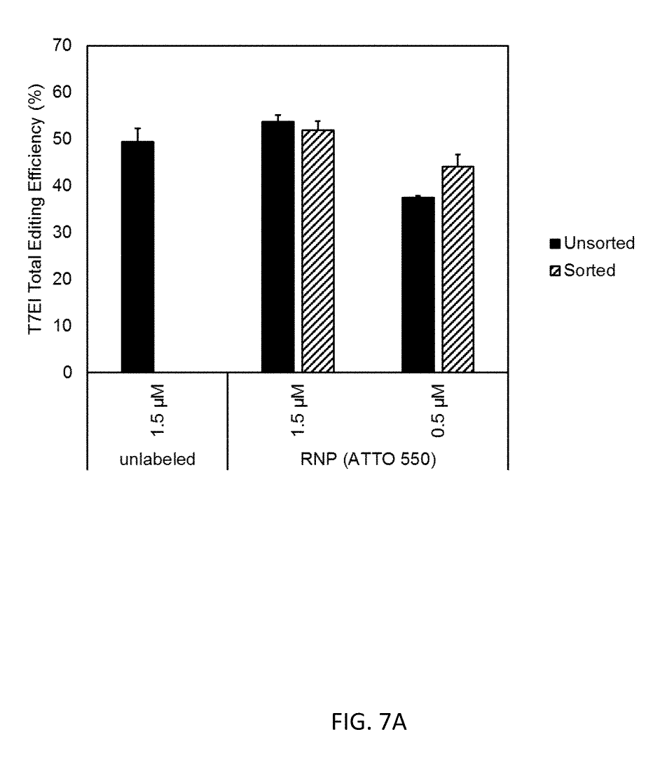

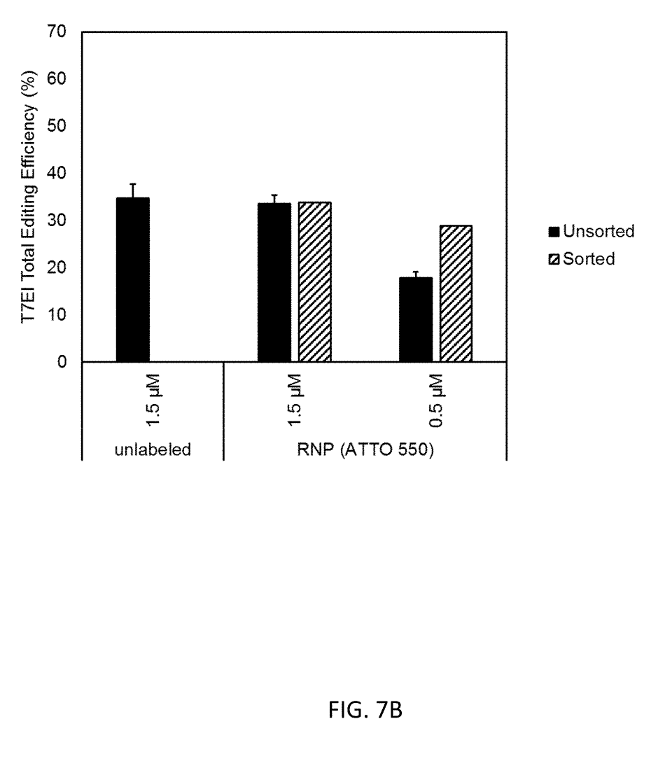

Enrichment of cells that contain the RNP will allow for increasing levels of edited cells. Fluorescence-activated cell sorting (FACS) was used to select cells that contain RNP consisting of a fluorescently-labeled tracrRNA. As shown in FIG. 4, the sorting gates were set to use the non-electroporated cells to eliminate the false-positive cell population caused by non-specific binding of the fluorescent dye to the lipid membrane. Levels of genome editing of unsorted and positively sorted were compared in both Jurkat cells (FIG. 7A) and HEK293 cells (FIG. 7B). At RNP concentrations of 1.5 the editing efficiencies of unsorted labeled cells are similar to unlabeled cells, showing that fluorescent labeling does not affect overall genome editing levels. Furthermore, the amount of editing in the positively sorted fractions is similar to the unsorted fractions when a concentration of 1.5 μM is used. These results show that maximum editing efficiencies are reached. However, when suboptimal RNP concentrations are used (0.5 an increase in genome editing levels is observed indicating that enrichment of cells that contain the RNP took place. Thus, delivery of labeled tracrRNA can lead to higher levels of genome editing by enrichment using FACS.

Jurkat cells were transfected using the Neon Transfection system (1E6 cells/1600V/10 ms/3pulses/100 μL cuvette tips) targeting the HPRT1 gene at position 38285-AS. Guide RNA complexes were made with unlabeled crRNA (SEQ ID NO:2) and unlabeled tracrRNA (SEQ ID NO:3) or ATTO550-labeled tracrRNA (SEQ ID NO:13). RNP concentrations of 1.5 and 0.5 μM were used. Alt-R® Cas9 Electroporation Enhancer was at a final concentration of 1.8 μM. After electroporation, cells were added to 1 mL of pre-warmed culture media in 12-well culture plates. 24 hours post transfection, the cells were split into two fractions; one unsorted fraction, and one fractions for FACS. The fractions for FACS analysis was washed once with 1× PBS. Following sorting, the positive and negative fractions were cultured for an additional 48 hours by incubation at 37° C. containing 5% CO₂. Genomic DNA was isolated from unsorted and sorted fractions, and the targeted genomic locus was amplified using PCR (SEQ ID Nos. 5,6) Heteroduplexes were formed by denaturing the amplicons followed by a slow cool-down. Mismatches in heteroduplexes were cleaved by 2.5 U T7 Endonuclease I, and cleaved and non-cleaved products were quantified using a Fragment Analyzer.

HEK293 cells were transfected using the Neon Transfection system (2.5E5 cells/1400V/10ms/3pulses/100 μL cuvette tips) targeting the HPRT 1 gene at position 38285-AS. Guide RNA complexes were made with unlabeled crRNA (SEQ ID NO:2) and unlabeled tracrRNA (SEQ ID NO:3) or ATTO550-labeled tracrRNA (SEQ ID NO:13). RNP concentrations of 1.5 and 0.5 μM were used. ^{AltR}® Cas9 Electroporation Enhancer was at a final concentration of 1.8 μM. After electroporation, cells were added to 1 mL of pre-warmed culture media in 12-well culture plates. 24 hours post transfection, the cells trypsinized, washed with 1× PBS, and split into two fractions; one unsorted fraction, and one fractions for FACS. The fractions for FACS analysis was washed once with 1× PBS. Following sorting, the positive and negative fractions were cultured for an additional 48 hours by incubation at 37° C. containing 5% CO₂. Genomic DNA was isolated from unsorted and sorted fractions, and the targeted genomic locus was amplified using PCR (SEQ ID Nos. 5,6) Heteroduplexes were formed by denaturing the amplicons followed by a slow cool-down. Mismatches in heteroduplexes were cleaved by 2.5 U T7 Endonuclease I, and cleaved and non-cleaved products were quantified using a Fragment Analyzer.

### EXAMPLE 5

The following example demonstrates that the labeled compositions and methods of the present invention allow for visualization of cell uptake through fluorescence microscopy.

A HEK293 cell line having constitutive expression of SpyCas9 (human codon- optimized) with stable vector integration and selection under G418 was developed as described below. Human optimized Spy Cas9 was ligated into a pcDNA3.1 expression vector (Life Technologies) and transfected into HEK293 cells using Lipofectamine2000 (Life Technologies). The transfected cells were allowed to grow for 2 days before being placed under selective pressure using Neomycin. After 7 days, cells were plated to single colonies using limiting dilution techniques. Monoclonal colonies were screened for Cas9 activity and the clone having highest level of expression was used for future studies.

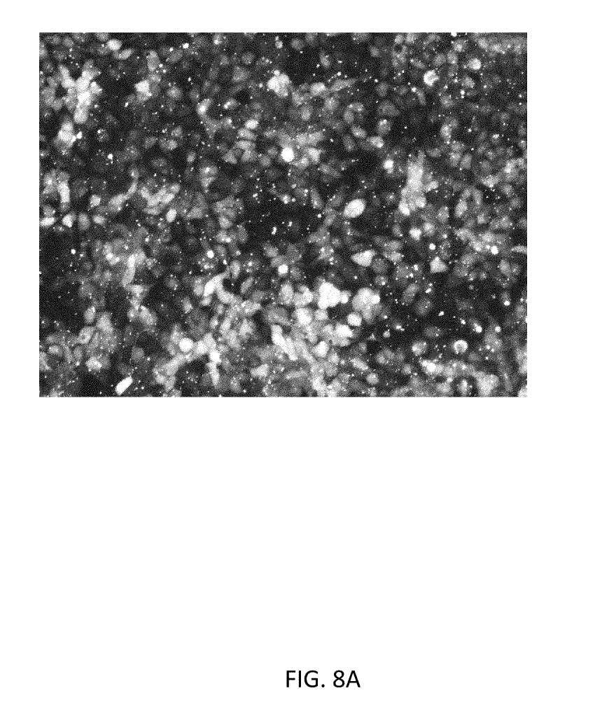

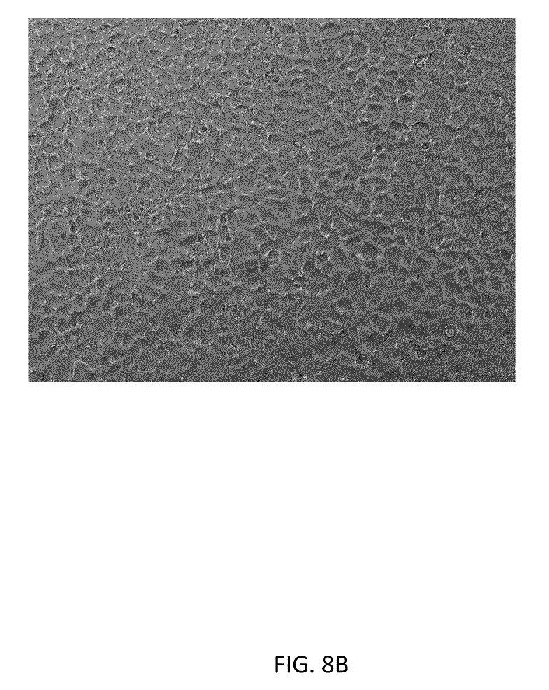

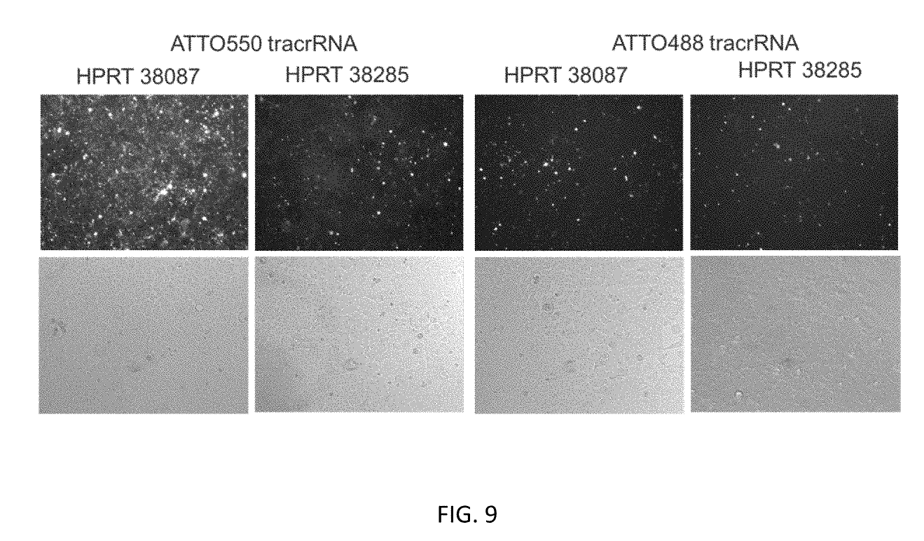

Fluorescently-labeled tracrRNA can also be used for visualization/localization purposes. The HEK293 cells that stably express Cas9 protein were transfected via lipofection with a guide RNA complex consisting of unlabeled crRNA and ATTO550-labeled tracrRNA. FIG. 8A shows that the fluorescently-labeled tracrRNA can be visualized intracellularly. Furthermore, this image also shows the relatively efficient uptake of the labeled guide RNA complex. FIG. 8B shows the same field as FIG. 8A, but under bright field conditions. Additionally, HEK293 cells were electroporated with RNPs that consist of unlabeled crRNA and ATTO550-labeled or ATTO488-labeled tracrRNA. The crRNAs were designed to target the HPRT1 gene either at position 38285-AS or at 38087-AS. FIG. 9 shows the intracellular detection of the labeled guide RNA complex (upper panels) for each fluorophore and each target site. Below the fluorescent images are the bright field images of the corresponding field (lower panels).

For lipofection experiments, the HEK293 cells stably expressing S.p. Cas9 were transfected using RNAiMAX. The guide RNA complex was formed by hybridization of equal molar amounts of unlabeled crRNA (SEQ ID NO:2) and ATTO550 labeled tracrRNA (SEQ ID NO:13) at a final concentration of 100 μM in IDT Duplex Buffer (30 mM HEPES, pH 7.5, 100 mM Potassium Acetate). The crRNA was specific to the HPRT1 gene at position 38285-AS (SEQ ID NO:2). The gRNA complex was incubated with 0.75 μL RNAiMAX in 50 μL of OptiMEM and then added to 4E4 HEK293-Cas9 expressing cells diluted in 100 μL growth media. Cells were washed with 1× PBS prior to imaging. Light and fluorescence microscopy images were taken after 48 hours.

For electroporation experiments, HEK293 cells were electroporated using the Amaxa Nucleofector System (Lonza). The guide RNA complex was formed by hybridization of equal molar amounts of crRNA and tracrRNA at a final concentration of 30 μM in IDTE. The unlabeled crRNA was specific to the HPRT1 gene at position 38285-AS (SEQ ID NO:2) or at position 38087-AS (SEQ ID NO:12). Either ATTO550-labeled tracrRNA (SEQ ID No.13), or ATTO-488-labeled tracrRNA (SEQ ID NO:17) was used. The ribonucleoprotein complex (RNP) was generated by complexation of equal molar amounts of Cas9 protein and guide RNA complex prior to electroporation in HEK293 cells. Cells were washed with 1× PBS prior to imaging. Light and fluorescence microscopy images were taken after 48 hours.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of detecting cells containing a CRISPR ribonucleoprotein complex, the method comprising:
a) contacting a CRISPR-associated RNA and a CRISPR-associated protein with a cell sample, wherein the CRISPR-associated RNA is conjugated to a label configured to generate a signal and the CRISPR-associated protein is Cas9 protein or Cpfl protein; and
b) determining a presence of the signal from at least one cell in the cell sample, wherein the presence of the signal from at least one cell in the cell sample detects at least one cell containing the CRISPR ribonucleoprotein complex.

2. The method of claim 1, further comprising enriching cells containing the CRISPR ribonucleoprotein complex from the cell sample using fluorescence-activated cell sorting.

3. The method according to claim 1, wherein the determining the presence of the signal from at least one cell in the cell sample comprises using fluorescence microscopy or fluorescence-activated cell sorting.

4. The method according to claim 1, wherein the CRISPR-associated RNA is selected from a gRNA for Cas9 protein or a crRNA for the Cpfl protein.

5. The method of claim 4, wherein the CRISPR-associated RNA is a gRNA for Cas9 protein, wherein the gRNA comprises a crRNA and a tracrRNA, further wherein the tracrRNA is conjugated to a label comprising a fluorescent dye.

6. The method of claim 4, wherein the CRISPR-associated RNA is a crRNA for the Cpfl protein, further wherein the crRNA is conjugated to a label comprising a fluorescent dye.)

7. The method according to claim 5, wherein the fluorescent dye is hydrophilic.

8. The method according to claim 5, wherein the fluorescent dye is a zwitterionic dye or a rhodamine-based dye selected from the group consisting of rhodamine 6G and rhodamine B.

9. The method according to claim 5, wherein the fluorescent dye is selected from the group consisting of ATTO550 dye, ATTO647 dye and ATTO488 dye.

10. A nucleic acid conjugate comprising a CRISPR-associated RNA conjugated to a label configured to generate a signal, wherein the CRISPR-associated RNA is selected from the group consisting of a crRNA and a tracrRNA.

11. The nucleic acid conjugate of claim 10, wherein the CRISPR-associated RNA is conjugated to a label comprising a fluorescent dye.

12. The nucleic acid conjugate of claim 11, wherein the fluorescent dye is hydrophilic.

13. The nucleic acid conjugate of claim 11, wherein the fluorescent dye is a zwitterionic dye or a rhodamine-based dye selected from the group consisting of rhodamine 6G and rhodamine B.

14. The nucleic acid conjugate of claim 11, wherein the fluorescent dye is selected from the group consisting of ATTO550 dye, ATTO647 dye and ATTO488 dye.

15. A kit for detecting cells containing a CRISPR ribonucleoprotein complex, the kit comprising a nucleic acid conjugate comprising a CRISPR-associated RNA conjugated to a label,
wherein the CRISPR-associated RNA is selected from the group consisting of a crRNA and a tracrRNA,
wherein the label comprises a fluorescent dye selected from the group consisting of ATTO550 dye, ATTO647 dye and ATTO488 dye.

16. The kit of claim 15, further comprising a positive cell control, wherein the positive cell control includes a CRISPR ribonucleoprotein complex comprising a Cas9 protein or a Cpfl protein and a nucleic acid conjugate comprising a CRISPR-associated RNA conjugated to a label,
wherein the CRISPR-associated RNA is selected from the group consisting of a crRNA and a tracrRNA,
wherein the label comprises a fluorescent dye selected from the group consisting of ATTO550 dye, ATTO647 dye and ATTO488 dye.

17. The kit of claim 16, further comprising a negative cell control, wherein the negative cell control includes a CRISPR ribonucleoprotein complex comprising a Cas9 protein or a Cpfl protein and an unlabeled CRISPR-associated RNA is selected from the group consisting of a crRNA and a tracrRNA.
